(19) United States
(12) Patent Application Publication
(10) Pub. No.: US 2014/0141473 A1
(43) Pub. Date: May 22, 2014

(54) YEAST CELL CAPABLE OF CONVERTING SUGARS INCLUDING ARABINOSE AND XLOSE

(71) Applicants: Paul Klaassen, Echt (NL); Bianca Elisabeth Maria Gielesen, Echt (NL); Gijsberdina Pieternella Van Suylekom, Echt (NL); Panagiotis Sarantinopoulos, Echt (NL); Wilbert Herman Marie Heijne, Echt (NL); Aldo Greeve, Echt (NL)

(72) Inventors: Paul Klaassen, Echt (NL); Bianca Elisabeth Maria Gielesen, Echt (NL); Gijsberdina Pieternella Van Suylekom, Echt (NL); Panagiotis Sarantinopoulos, Echt (NL); Wilbert Herman Marie Heijne, Echt (NL); Aldo Greeve, Echt (NL)

(73) Assignee: DSM IP ASSETS B.V., Heerlen (NL)

(21) Appl. No.: 14/112,713

(22) PCT Filed: Apr. 20, 2012
(86) PCT No.: PCT/EP2012/057273

§ 371 Date: Dec. 16, 2013

Related U.S. Application Data
(60) Provisional application No. 61/478,142, filed on Apr. 22, 2011.

(30) Foreign Application Priority Data
Apr. 22, 2011 (EP) 11163579.3

(51) Int. Cl. C12P 7/10

(52) U.S. Cl. CPC C12P 7/10
USPC 435/108; 435/106; 435/109; 435/113; 435/115; 435/136; 435/139; 435/140; 435/142; 435/144; 435/145; 435/158; 435/159; 435/160; 435/165; 435/167; 435/171; 435/189; 435/193; 435/195; 435/198; 435/201; 435/209; 435/219; 435/232; 435/254.21

(57) ABSTRACT

Yeast cell belonging to the genus Saccharomyces having introduced into its genome at least one xylA gene and at least one of each of araA, araB and araD genes and that is capable of consuming a mixed sugar mixture comprising glucose, xylose and arabinose, wherein the cell co-consumes glucose and arabinose, has genetic variations obtained during adaptive evolution and has a specific xylose consumption rate in the presence of glucose that is 0.25 g xylose/h, g DM or more.

### FIELD OF THE INVENTION

The invention relates to yeast cells which are capable of converting sugars including arabinose and xylose. The invention further relates to a process in which such cells are used for the production of a fermentation product, such as ethanol.

### BACKGROUND OF THE INVENTION

Large-scale consumption of traditional, fossil fuels (petroleum-based fuels) in recent decades has contributed to high levels of pollution. This, along with the realisation that the world stock of fossil fuels is limited and a growing environmental awareness, has stimulated new initiatives to investigate the feasibility of alternative fuels such as ethanol, which is a particulate-free burning fuel source that releases less CO2 than unleaded gasoline on a per litre basis. Although biomass-derived ethanol may be produced by the fermentation of hexose sugars obtained from many different sources, the substrates typically used for commercial scale production of fuel alcohol, such as cane sugar and corn starch, are expensive. Increases in the production of fuel ethanol will therefore require the use of lower-cost feedstocks. Currently, only lignocellulosic feedstock derived from plant biomass is available in sufficient quantities to substitute the crops currently used for ethanol production. In most lignocellulosic material, next to C6 sugar also contain considerable amounts of C5 sugars, including arabinose and xylose. Thus, for an economically feasible fuel production process, both hexose and pentose sugars must be fermented to form ethanol. The yeast Saccharomyces cerevisiae is robust and well adapted for ethanol production, but it is unable to convert arabinose and xylose. Also, no naturally-occurring organisms are known which can ferment xylose or arabinose to ethanol with both a high ethanol yield and high ethanol productivity. There is therefore a need for an organism possessing these properties so as to enable the commercially-viable production of ethanol from lignocellulosic feedstocks. In co-pending patent application that has not been published at the date of filing of this application (EP10160647.3 and the PCT application claiming its priority), strain BIE252 is described. This strain is able to ferment a mixed sugar composition that includes glucose, xylose, arabinose, galactose, and mannose and to produce fermentation product. Strain BIE252 is able to convert all these sugars, but for most part, glucose is consumed first, and the other sugars thereafter, it would be desirable to have co-consumption of glucose and C5-sugars including arabinose and xylose, since it maybe expected that shorter fermentation times are then possible.

### SUMMARY OF THE INVENTION

An object of the invention is to provide a cell, in particular a yeast cell that is capable of converting a mixed sugar composition that comprises glucose, xylose and arabinose. Another object is to provide such cell that converts a mixed sugar composition that comprises glucose, xylose and arabinose in high yield. Another object is to provide such cell that is able to co-consume C5 and C6 sugar. Another object is to provide such cell, that has a high productivity. A further object is to provide such cell that is genetically stable. One or more of these objects is attained according to the invention that provides A yeast cell belonging to the genus Saccharomyces having introduced into its genome at least one xylA gene and at least one of each of araA, araB and araD genes and that is capable of consuming a mixed sugar mixture comprising glucose, xylose and arabinose, wherein the cell co-consumes glucose and arabinose, has genetic variations obtained during adaptive evolution and has a specific xylose consumption rate in the presence of glucose that is 0.25 g xylose/h, g DM or more.

The yeast cell of the invention is capable of converting a mixed sugar composition that comprises glucose, xylose and arabinose in high yield. Further the yeast cell has a high productivity as defined hereinafter. This allows a reduction of fermentation time. Additionally the yeast cell is genetically stable. The latter is advantageous when the yeast is used in an industrial process.

In an embodiment the yeast cell is Saccharomyces cerevisiae.

### BRIEF DESCRIPTION OF THE DRAWINGS

FIG. 1 sets out the growth rates on arabinose and xylose after each cycle in the SBR cultivation system of the improved cultures of strain S. cerevisiae BIE252.

FIG. 2 sets out the sugar conversion and product formation of strain BIE252 on synthetic medium, in the BAM system. CO2 production was measured constantly. Growth was monitored by following optical density of the culture. Preculture was grown on 2% glucose.

FIG. 3 sets out the sugar conversion and product formation of strain BIE272 on synthetic medium, in the BAM system. CO2 production was measured constantly. Growth was monitored by following optical density of the culture. Preculture was grown on 2% glucose.

FIG. 4 sets out the xylose consumption by strains BIE252 and BIE272 in the AFM fermentations in real hydrolysates at 10 and 20% dry matter pCS.

FIG. 5 sets out the arabinose consumption by strains BIE252 and BIE272 in the AFM fermentations in real hydrolysates at 10 and 20% dry matter pCS.

FIG. 6 sets out the ethanol that was produced by strains BIE252 and BIE272 in the AFM fermentations in real hydrolysates at 10 and 20% dry matter pCS.

FIG. 7 sets out the CO₂ that was produced by strains BIE252 and BIE272 in the AFM fermentations in real hydrolysates at 10 and 20% dry matter pCS.

FIG. 8 sets out the performance of strain BIE272 in pretreated, hydrolyzed corn stover at 20% dry matter. Ethanol production and sugar conversion are shown.

FIG. 9 sets out the performance stability of strain BIE272. Two colonies isolated directly from the glycerol stock of strain BIE272 and six colonies after cultivation in YEP 2% glucose for 10, 19, 28, 37 and 46 generations were tested for their ability to grow on Verduyn medium supplemented with 2% xylose. The grey parts of the bars represent the number of colonies exhibiting xylose growth better than or equal to the reference strain, BIE272. The black parts of the bar indicate the number of colonies that are lagging behind. The experiment was performed in duplicate. The left panel represents the results of shake flask 1, the right panel the results of shake flask 2.

FIG. 10 sets out a CHEF gel, stained with ethidium bromide. Chromosomes were separated on their size using the CHEF technique. Strains analyzed are BIE104; BIE104A2P1a, (synonym of BIE104A2P1); BIE104A2P1c; strain BIE201; BIE201X9; BIE252 and BIE272. Shifts in chromosomes are observed (see text). Strain YNN295 is a marker strain (Bio-Rad), used as a reference for the size of the chromosomes.

FIG. 11 sets out an autoradiogram of a CHEF gel, blotted to a membrane and hybridized with a PNC1 probe. Strains analyzed are BIE104; BIE104A2P1a, (synonym of BIE104A2P1); BIE104A2P1c; strain BIE201; BIE201X9; BIE252 and BIE272. Shifts in chromosomes are observed (see text).

FIG. 12 sets out an autoradiogram of a CHEF gel, blotted to a membrane and hybridized with a ACT1 probe (left panel, a) and the xylA probe (right panel, b). Strains analyzed are BIE104; BIE104A2P1a, (synonym of BIE104A2P1); BIE104A2P1c; strain BIE201; BIE201X9; BIE252 and BIE272. Shifts in chromosomes are observed (see text).

FIG. 13 sets out the CO₂ production rate (in ml CO₂ per minute) of strains BIE104, BIE201, BIE252 and BIE272.

FIG. 14 sets out the CO₂ production rate (in ml CO₂ per minute) of strains BIE104 and BIE201.

FIG. 15 sets out the CO₂ production rate (in ml CO₂ per minute) of strains BIE201 and BIE252.

FIG. 16 sets out the CO₂ production rate (in ml CO₂ per minute) of strains BIE252 and BIE272.

FIG. 17 sets out the sugar conversion and product formation of strain BIE104 on synthetic medium, in the BAM system. CO2 production was measured constantly. Growth was monitored by following optical density of the culture.

FIG. 18 sets out the sugar conversion and product formation of strain BIE201 on synthetic medium, in the BAM system. CO2 production was measured constantly. Growth was monitored by following optical density of the culture.

FIG. 19 sets out the sugar conversion and product formation of strain BIE252 on synthetic medium, in the BAM system. CO2 production was measured constantly. Growth was monitored by following optical density of the culture.

FIG. 20 sets out the sugar conversion and product formation of strain BIE272 on synthetic medium, in the BAM system. CO2 production was measured constantly. Growth was monitored by following optical density of the culture.

FIG. 21 sets out the normalized read depth (or coverage) of the PMA1-gene.

FIG. 22 sets out the the normalized read depth (or coverage) of the xylA gene.

### BRIEF DESCRIPTION OF THE SEQUENCE LISTING

SEQ ID NO: 1: synthetic DNA, forward primer xylA, CACCGTTAGCCTTGGCGTAAGC

SEQ ID NO: 2 synthetic DNA, reverse primer xylA, CACTTTCGAACACGAATTGGC

SEQ ID NO: 3 synthetic DNA, forward primer ACT1, GTTACGTCGCCTTGGACTTCG

SEQ ID NO: 4 synthetic DNA, reverse primer ACT1, CGGCAATACCTGGGAACATGG

SEQ ID NO: 5 synthetic DNA, forward primer PNC1, GATAGAGACTGGCACAGGATTG

SEQ ID NO: 6 synthetic DNA, reverse primer PNC1, ACAATACTCCAAAGCTACACC

SEQ ID NO: 7 wild-type PMR1 protein sequence

SEQ ID NO: 8 PMR1 protein sequence of yeast strain BIE272

### DETAILED DESCRIPTION OF THE INVENTION

Throughout the present specification and the accompanying claims, the words “comprise” and “include” and variations such as “comprises”, “comprising”, “includes” and “including” are to be interpreted inclusively. That is, these words are intended to convey the possible inclusion of other elements or integers not specifically recited, where the context allows.

The articles “a” and “an” are used herein to refer to one or to more than one (i.e. to one or at least one) of the grammatical object of the article. By way of example, “an element” may mean one element or more than one element.

Yeast cell or yeast cells may herein also called yeast strain.

The various embodiments of the invention described herein may be cross-combined.

The invention relates to a yeast cell belonging to the genus Saccharomyces having introduced into its genome at least one xylA gene and at least one of each of araA, araB and araD genes and that is capable of consuming a mixed sugar mixture comprising glucose, xylose and arabinose, wherein the cell co-consumes glucose and arabinose, and the specific xylose consumption rate in the presence of glucose is 0.25 g xylose/h, g DM or more. Herein DM is dry yeast biomass.

In an embodiment, the specific xylose consumption rate in the presence of glucose is 0.25 or more, 0.30 or more, 0.35 or more. 0.40 or more, or about 0.41 g xylose/h, g DM. In an embodiment the the specific xylose consumption rate in the presence of glucose is 0.25 to 0.60 g arabinose/h/g DM. In an embodiment, the yeast cell wherein the copy number of the araA, araB and araD genes is three or four each. In another embodiment the yeast cell has a copy number of xylA of about 9 or 10.

In another embodiment, the yeast cell has one or more of the single nucleotide polymorphism chosen from the group consisting of mutations G1363T in the SSY1 gene, A512T in YJR154w gene, A1186G in CEP3 gene, A436C in GAL80 gene and A113G in PMR1 gene.

In an embodiment the yeast cell has a single polymorphism A436C in GAL80 gene. Optionally it also has a single nucleotide polymorphism A1186G in CEP3 gene or a single nucleotide polymorphism A113G in PMR1 gene.

In an embodiment, the yeast cell has a yield of 0.40 g ethanol/g sugar or more or about 0.42. In another embodiment, the yeast cell has a productivity of 1.20 or more g EtOH/I, h. In an embodiment the yeast cell has a productivity of 1.25 or more, 1.30 or more, 1.35 or more, 1.40 or more, 1.45 or more, 1.50 or more, 1.55 or more, 1.60 or more or 1.65 or more g EtOH/I, h. In an embodiment the yeast cell has a productivity of about 1.69 g EtOH/I, h. The productivity is herein measured in the interval of 0-24 h after start of the fermentation. Also in other time intervals the productivity of the yeast cells is high. See table 11.

The invention further relates to a polypeptide having aminoacid sequence of SEQ ID NO: 7 having a substitution Tyr38Cys in PMR1, resulting in SEQ ID NO: 8; and variant polypeptides thereof wherein one or more of the other positions may have mutation of the aminoacid with an aminoacid that is an existing conserved aminoacid in the SPCA (Secretory Pathway calcium ATP-ase) family. The invention further relates to a process for the production of one or more fermentation products from a sugar composition comprising glucose, xylose, arabinose galactose and mannose and wherein the sugar composition is fermented with a yeast cell according to the invention. In an embodiment of the process, the sugar composition is produced from lignocellulosic material by: pretreatment of one or more lignocellulosic material to produce pretreated lignocellulosic material; enzymatic treatment of the pretreated lignocellulosic material to produce the sugar composition.

In another embodiment, in the process, the fermentation is conducted anaerobically. The fermentation product may be selected from the group consisting of ethanol, n-butanol, isobutanol, lactic acid, 3-hydroxy-propionic acid, acrylic acid, acetic acid, succinic acid, fumaric acid, malic acid, itaconic acid, maleic acid, citric acid, adipic acid, an amino acid, such as lysine, methionine, tryptophan, threonine, and aspartic acid, 1,3-propane-diol, ethylene, glycerol, a β-lactam antibiotic and a cephalosporin, vitamins, pharmaceuticals, animal feed supplements, specialty chemicals, chemical feedstocks, plastics, solvents, fuels, including biofuels and biogas or organic polymers, and an industrial enzyme, such as a protease, a cellulase, an amylase, a glucanase, a lactase, a lipase, a lyase, an oxidoreductases, a transferase or a xylanase.

In an embodiment, the yeast cell has a chromosome that is amplified compared to the host strain, wherein the amplified chromosome has the same number as the chromosome in which the araA, araB and araD genes were introduced in the host strain. In an embodiment the amplified chromosome is chromosome VII. In an embodiment, in the yeast cell parts of chromosome VII, surrounding the centromere, are amplified (as compared to the host strain). In an embodiment, part of the right arm of chromosome VII was amplified twice, and an adjacent part was amplified three times.

The part on the right arm of chromosome VII that was amplified three times contains the arabinose expression cassette, i.e. the genes araA, araB and araD under control of strong constitutive promoters.

The invention further relates to a yeast cell having araA, araB and araD genes wherein chromosome VII has a size of from 1300 to 1400 kb, or 1375 kb as determined by electrophoresis as measured as described hereinafter.

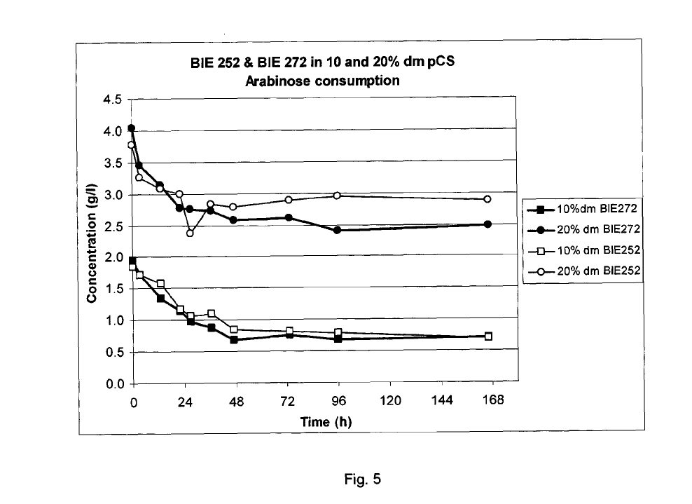

In an embodiment, in the yeast cell, the copy number of the araA, araB and araD genes is two to ten, in an embodiment two to eight or three to five each. The copy number of the araA, araB and araD genes may be 2, 3, 4, 5, 6, 7, 8, 9, or 10. The copy number may be determined with methods known to the skilled person, Suitable methods are illustrated in the examples, and results are e.g. shown in FIG. 5.

In an embodiment, the yeast cell one or more of the single nucleotide polymorphism chosen from the group consisting of mutations G1363T in the SSY1 gene, A512T in YJR154w gene, A1186G in CEP3 gene, A436C in GAL80 gene and A113G in PMR1. In an embodiment, the yeast cell has a single polymorphism A436C in GAL80 gene. In an embodiment, the yeast cell has a single polymorphism A1186G in CEP3 gene. In an embodiment, the yeast cell has a single polymorphism A113G in PMR1.

Adaptation

Adaptation is the evolutionary process whereby a population becomes better suited (adapted) to its habitat or habitats. This process takes place over several to many generations, and is one of the basic phenomena of biology.

The term adaptation may also refer to a feature which is especially important for an organism's survival. Such adaptations are produced in a variable population by the better suited forms reproducing more successfully, by natural selection.

Changes in environmental conditions alter the outcome of natural selection, affecting the selective benefits of subsequent adaptations that improve an organism's fitness under the new conditions. In the case of an extreme environmental change, the appearance and fixation of beneficial adaptations can be essential for survival. A large number of different factors, such as e.g. nutrient availability, temperature, the availability of oxygen, etcetera, can drive adaptive evolution.

For example, a haploid yeast strain, transformed with genes necessary for or enhancing the ability to ferment arabinose (designated all together as ARA) was enhanced by a process called adaptive evolution. During the adaptive evolution process, three mutations have been introduced into the genome, designated mut1, mut2 and mut3. The genotype of such a yeast strain could be written as mut1 mut2 mut3 ARA.

Fitness

There is a clear relationship between adaptedness (the degree to which an organism is able to live and reproduce in a given set of habitats) and fitness. Fitness is an estimate and a predictor of the rate of natural selection. By the application of natural selection, the relative frequencies of alternative phenotypes will vary in time, if they are heritable.

Genetic Changes/Variations

When natural selection acts on the genetic variability of the population, genetic changes are the underlying mechanism. By this means, the population adapts genetically to its circumstances. Genetic changes may result in visible structures, or may adjust the physiological activity of the organism in a way that suits the changed habitat.

It may occur that habitats frequently change. Therefore, it follows that the process of adaptation is never finally complete. In time, it may happen that the environment changes gradually, and the species comes to fit its surroundings better and better. On the other hand, it may happen that changes in the environment occur relatively rapidly, and then the species becomes less and less well adapted. Adaptation is a genetic process, which goes on all the time to some extent, also when the population does not change the habitat or environment.

Single nucleotides in a DNA sequence may be changed (substitution), removed (deletions) or added (insertion). Insertion or deletion SNPs (InDels) may shift the translational frame.

Single nucleotide polymorphisms may fall within coding sequences of genes (Open Reading Frames or ORFs), non-coding regions of genes (like promoter sequences, terminator sequences and the like), or in the intergenic regions between genes. SNPs within a coding sequence will not necessarily change the amino acid sequence of the corresponding protein that is produced after transcription and translation, due to degeneracy of the genetic code. A SNP in which both forms lead to the same polypeptide sequence is termed synonymous (a silent mutation). If a different polypeptide sequence is produced they are nonsynonymous. A nonsynonymous change may either be missense or nonsense. A missense change results in a different amino acid in the corresponding polypeptide, while a nonsense change results in a premature stop codon, sometimes leading to the formation of a truncated protein.

SNPs that are not in protein-coding regions may still have consequences for gene expression, for instance by a changed transcription factor binding or stability of the corresponding mRNA.

The changes that may occur in the DNA are not necessarily limited to the change (substitution, deletion or insertion) of a single nucleotide, but may also comprise a change of two or more nucleotides (Small Nuclear Variations).

In addition, chromosomal translocations may occur. A chromosome translocation is a chromosome abnormality caused by rearrangement of parts between nonhomologous chromosomes.

In particular, in the cell according to the invention SNP are created in the following reading frames: SSY1, CEP3, GAL80 and PMR1.

SSY1 is herein a component of the SPS plasma membrane amino acid sensor system (Ssy1p-Ptr3p-Ssy5p), which senses external amino acid concentration and transmits intracellular signals that result in regulation of expression of amino acid permease genes.

CEP3 is herein an essential kinetochore protein, component of the CBF3 complex that binds the CDEIII region of the centromere; contains an N-terminal Zn2Cys6 type zinc finger domain, a C-terminal acidic domain, and a putative coiled coil dimerization domain.

GAL80 is herein a transcriptional regulator involved in the repression of GAL genes in the absence of galactose. Typically it inhibits transcriptional activation by Gal4p and inhibition is relieved by Gal3p or Gal1p binding.

PMR1 (systematic name YGL167c) is herein a High affinity Ca2+/Mn2+P-type ATPase required for Ca2+ and Mn2+ transport into Golgi; involved in Ca2+ dependent protein sorting and processing. Pmr1p is the prototype of a family of transporters known as SPCA (Secretory Pathway Ca2+-ATPases) with members found in fungi, C. elegans, D. melanogaster, and mammals.

According to the invention, SNP's in the genes SSY1, CEP3, GAL80 and PMR1 have been shown to be important for the cell to be able to ferment a mixed sugar composition.

BLAST searches were conducted for the SNP's found in these genes.

An overview of the SNP that were identified is given in table 1:

TABLE 1
Overview of SNP's

| | Nucleotide mutation | Amino acid mutation |
|---|---|---|
| Gene | position in ORF* | position in protein |
| SSY1 | G1363T | E455stop |
| YJR154w | A512G | D171G |
| CEP3 | A1186G | S396G |
| GAL80 | A436C | T146P |
| PMR1 | A113G | Y38C |

*the A of the start codon ATG is the first nucleotide position

A blast of the genes containing the SNP resulted in the following data:

Ssy1p (Member of the AA Trans Superfamily)

Component of the SPS plasma membrane amino acid sensor system (Ssy1p-Ptr3p-Ssy5p), which senses external amino acid concentration and transmits intracellular signals that result in regulation of expression of amino acid permease genes [Saccharomyces cerevisiae]

| | | | |
|---|---|---|---|
| Ssy1p | S. cerevisiae JAY291 | 852 aa | 99% identity |
| Ssy1p | S. cerevisiae YJM789 | 852 aa | 99% identity |
| YDR160w-like | S. cerevisiae AWRI1631 | 791 aa | 99% identity |
| protein | | | |
| ZYRO0F13838p | Z. rouxii CBS 732 | 836 aa | 56% identity |
| hypothetical protein | C. glabrata CBS 138 | 853 aa | 53% identity |
| KLTH0G11726p | Lachancea thermotolerans | 824 aa | 46% identity |

Shorter protein found in S. cerevisiae BIE201 is a unique feature.

YJR154w (Member of the PhyH Superfamilv)

Putative protein of unknown function; green fluorescent protein (GFP)-fusion protein localizes to the cytoplasm [Saccharomyces cerevisiae]

| | | | |
|---|---|---|---|
| YJR154w | S. cerevisiae JAY291 | 346 aa | 100% identity |
| conserved protein | S. cerevisiae YJM789 | 346 aa | 99% identity |
| putative pimeloyl- | S. cerevisiae | 346 aa | 71% identity |
| CoA synth. | | | |
| YJR154Wp-like | S. cerevisiae AWRI1631 | 227 aa | 99% identity |
| protein | | | |
| KLTH0E09900p | Lachancea thermotolerans | 340 aa | 48% identity |

In all these proteins, the D-residue at position 171 (or equivalent position based on the BLAST results) is conserved.

CEP3 (GAL4-Like Zn2Cys6 Binuclear Cluster DNA-Binding Domain; Found in Transcription Regulators Like GAL4)

Centromere DNA-Binding Protein Complex CBF3 Subunit B

| | | | |
|---|---|---|---|
| CEP3 | S. cerevisiae JAY291 | 608 aa | 100% identity |
| ZYRO0A07260p | Z. rouxii CBS 732 | 596 aa | 46% identity |
| unnamed protein | Candida glabrata CBS138 | 611 aa | 44% identity |
| product | | | |
| AFL200Wp | A. gossypii ATCC 10895 | 596 aa | 41% identity |

In all these proteins, the S-residue at position 396 (or equivalent position based on the BLAST results) is conserved.

GAL80 (Member of the NADB Rossmann Superfamily)

Galactose/Lactose Metabolism Regulatory Protein GAL80

| | | | |
|---|---|---|---|
| transcriptional | S. cerevisiae | 435 aa | 100% |
| regulator | YJM789 | | identity |
| GAL80p | S. kudriavzevii | 435 aa | 89% |
| | | | identity |
| protein Kpol_1059p5 | V. polyspora | 429 aa | 73% |
| | DSM 70294 | | identity |
| ZYRO0G04664p | Z. rouxii CBS 732 | 437 aa | 67% |
| | | | identity |
| KLTH0C02838p | L. thermotolerans | 424 aa | 64% |
| | | | identity |
| KIGAL80 protein | Kluyveromyces lactis | 457 aa | 58% |
| | | | identity |
| NECHADRAFT_86878 | N. haematococca | 367 aa | 30% |
| | mpVI 77-13-4 | | identity |

In all these proteins, the T-residue at position 146 (or equivalent position based on the BLAST results) is conserved.

PMR1 from Strain BIE272 (Member of the Family SPCA (Secretory Pathway Ca2+-ATPases

| | | | |
|---|---|---|---|
| Pmr1p | S. cerevisiae S288c | 950 aa | 99% identity |
| Pmr1p | S. cerevisiae JAY291 | 950 aa | 99% identity |
| hypothetical protein | C. glabrata CBS 138 | 951 aa | 79% identity |
| ZYRO0E02860p | Z. rouxii | 943 aa | 75% identity |
| AEL301Wp | A. gossypii ATCC 10895 | 957 aa | 71% identity |
| Ca2+-pump ATPase | C. tropicalis MYA-3404 | 919 aa | 61% identity |

Structural Variations

Structural variation (also genomic structural variation) consists of many kinds of variation in the genome of one species, and usually includes microscopic and submicroscopic types, such as deletions, duplications, copy number variations, insertions, inversions and translocations.

Read Depth

The read depth (or coverage) represents the (average) number of nucleotides contributing to a portion of a Next Generation Sequencing assembly. The read depth expresses the number of times each base has been read. The read depth varies depending on the genomic region. The average read depth may also vary depending on the mapping criteria, such as stringency and read quality.

The average sequencing depth of genomic regions is compared between sequences. This allows for the detection of regions that are over- or underrepresented.

Copy Number Variation

Copy-number variation (CNV) is a large category of structural variations, which includes insertions, deletions and duplications.

Simile Nucleotide Polymorphism

A single nucleotide polymorphism (SNP) is a DNA sequence variation occurring when a single nucleotide—A, T, C, or G—in the genome (or other shared sequence) differs between members of a biological species or paired chromosomes in an individual cell.

Single nucleotide polymorphisms may fall within coding sequences of genes, non-coding regions of genes, or in the intergenic regions between genes. SNPs within a coding sequence do not necessarily change the amino acid sequence of the protein that is produced, due to degeneracy of the genetic code.

Indel or DIP

In evolutionary studies, indel is used to mean an insertion or a deletion. Indels refer to the mutation class that includes both insertions, deletions, and the combination thereof.

Pulsed Field Gel Electrophoresis (PFGE)

PFGE is a technique used for the separation of large deoxyribonucleic acid (DNA) molecules by applying an electric field that periodically changes direction to a gel matrix. PFGE can be performed using a variety of alternative systems, such as transverse Alternating Pulsed-Field Electrophoresis (TAFE), Orthogonal Field Alternation Gel Electrophoresis (OFAGE), Field Inversion Gel Electrophoresis (FIGE) and Contour-clamped Homogeneous Electric Field (CHEF) gel electrophoresis. Each method has its advantages and disadvantages with respect to ease of use, time required to perform the electrophoresis and the resolution of the chromosomes, as reviewed by Basim and Basim (Turk J Biol 25 (2001) 405-418) and references therein.

CHEF gel electrophoresis can produce substantial chromosomal separation of chromosomes from 100 to 2500 kb of Saccharomyces yeast strains on one gel, although not all the larger sized and similarly sized chromosomes resolve (Sheehan et al (1991) J. Inst. Brew., Vol. 97, 163-167).

### The Mixed Sugar Composition

The sugar composition according to the invention comprises glucose, arabinose and xylose. Any sugar composition may be used in the invention that suffices those criteria. Optional sugars in the sugar composition are galactose and mannose, and rhamnose. In a preferred embodiment, the sugar composition is a hydrolysate of one or more lignocellulosic material. Lignocellulose herein includes hemicellulose and hemicellulose parts of biomass. Also lignocellulose includes lignocellulosic fractions of biomass. Suitable lignocellulosic materials may be found in the following list: orchard primings, chaparral, mill waste, urban wood waste, municipal waste, logging waste, forest thinnings, short-rotation woody crops, industrial waste, wheat straw, oat straw, rice straw, barley straw, rye straw, flax straw, soy hulls, rice hulls, corn gluten feed, oat hulls, sugar cane, corn stover, corn stalks, corn cobs, corn husks, switch grass, miscanthus, sweet sorghum, canola stems, soybean stems, prairie grass, gamagrass, foxtail; sugar beet pulp, citrus fruit pulp, seed hulls, cellulosic animal wastes, lawn clippings, cotton, seaweed, trees, softwood, hardwood, poplar, pine, shrubs, grasses, wheat, wheat straw, sugar cane bagasse, corn, corn kernel, fiber from kernels, products and by-products from wet or dry milling of grains, municipal solid waste, waste paper, yard waste, herbaceous material, agricultural residues, forestry residues, pulp, paper mill residues, branches, bushes, canes, an energy crop, forest, a fruit, a flower, a grain, a grass, a herbaceous crop, a leaf, bark, a needle, a log, a root, a sapling, a shrub, switch grass, a tree, a vegetable, fruit peel, a vine, sugar beet pulp, wheat midlings, organic waste material generated from an agricultural process, forestry wood waste, or a combination of any two or more thereof. In an embodiment, the lignocellulosic material is from wheat, corn, sugar cane, rice or grass, e.g. corn stover, corn fiber, corn cobs, wheat straw, rice hulls, sugar cane bagasse or types of grass or other energy crops.

An overview of some suitable sugar compositions derived from lignocellulose and the sugar composition of their hydrolysates is given in table 2. The listed lignocelluloses include: corn cobs, corn fiber, rice hulls, melon shells, sugar beet pulp, wheat straw, sugar cane bagasse, wood, grass and olive pressings.

TABLE 2
Overview of sugar compositions from lignocellulosic materials. Gal = galactose,
Xyl = xylose, Ara = arabinose, Man = mannose, Glu = glutamate,
Rham = rhamnose. The percentage galactose (% Gal) and literature source is given.

| | Lignocellulosic material | | | | | | | | |
|---|---|---|---|---|---|---|---|---|---|
| | Gal | Xyl | Ara | Man | Glu | Rham | Sum | %. Gal. | Lit. |
| Corn cob a | 10 | 286 | 36 | | 227 | 11 | 570 | 1.7 | (1) |
| Corn cob b | 131 | 228 | 160 | | 144 | | 663 | 19.8 | (1) |
| Rice hulls a | 9 | 122 | 24 | 18 | 234 | 10 | 417 | 2.2 | (1) |
| Rice hulls b | 8 | 120 | 28 | | 209 | 12 | 378 | 2.2 | (1) |
| Melon Shells | 6 | 120 | 11 | | 208 | 16 | 361 | 1.7 | (1) |
| Sugar beet pulp | 51 | 17 | 209 | 11 | 211 | 24 | 523 | 9.8 | (2) |
| Wheat straw Idaho | 15 | 249 | 36 | | 396 | | 696 | 2.2 | (3) |
| Corn fiber | 36 | 176 | 113 | | 372 | | 697 | 5.2 | (4) |
| Cane Bagasse | 14 | 180 | 24 | 5 | 391 | | 614 | 2.3 | (5) |
| Corn stover | 19 | 209 | 29 | | 370 | | 626 | | (6) |
| Athel (wood) | 5 | 118 | 7 | 3 | 493 | | 625 | 0.7 | (7) |
| Eucalyptus (wood) | 22 | 105 | 8 | 3 | 445 | | 583 | 3.8 | (7) |
| CWR (grass) | 8 | 165 | 33 | | 340 | | 546 | 1.4 | (7) |
| JTW (grass) | 7 | 169 | 28 | | 311 | | 515 | 1.3 | (7) |
| MSW | 4 | 24 | 5 | 20 | 440 | | 493 | 0.9 | (7) |
| Reed Canary | 16 | 117 | 30 | 6 | 209 | 1 | 379 | 4.2 | (8) |
| Grass Veg | | | | | | | | | |
| Reed Canary | 13 | 163 | 28 | 6 | 265 | 1 | 476 | 2.7 | (9) |
| Grass Seed | | | | | | | | | |
| Olive pressing | 15 | 111 | 24 | 8 | 329 | | 487 | 3.1 | (9) |
| residue | | | | | | | | | |

It is clear from table 2 that in these lignocelluloses a high amount of sugar is presence in de form of glucose, xylose, arabinose and galactose. The conversion of glucose, xylose, arabinose and galactose to fermentation product is thus of great economic importance. Also mannose and rhamnose is present in some lignocellulose materials be it in lower amounts than the previously mentioned sugars. Advantageously therefore also mannose and rhamnose is converted by the mixed sugar cell.

Pretreatment and Enzymatic Hydrolysis

Pretreatment and enzymatic hydrolysis may be needed to release sugars that may be fermented according to the invention from the lignocellulosic (including hemicellulosic) material. These steps may be executed with conventional methods.

The Mixed Sugar Cell

The mixed sugar cell comprising the genes araA, araB and araD integrated into the mixed sugar cell genome as defined hereafter. It is able to ferment glucose, arabinose, xylose, galactose and mannose. In one embodiment of the invention the mixed sugar cell is able to ferment one or more additional sugar, preferably C5 and/or C6 sugar. In an embodiment of the invention the mixed sugar cell comprises one or more of: a xylA-gene and/or XKS1-gene, to allow the mixed sugar cell to ferment xylose; deletion of the aldose reductase (GRE3) gene; overexpression of PPP-genes TAK1, TKL1, RPE1 and RKI1 to allow the increase of the flux through the pentose phosphate pass-way in the cell.

Construction of the Mixed Sugar Strain

The genes may be introduced in the mixed sugar cell by introduction into a host cell:
a) a cluster consisting of PPP-genes TAD, TKL1, RPE1 and RKI1, under control of strong promoters;
b) a cluster consisting of a xylA-gene and a XKS1-gene both under control of constitutive promoters,
c) a cluster consisting of the genes araA, araB and araD and/or a cluster of xylA-gene and/or the XKS1-gene;
and
d) deletion of an aldose reductase gene
and adaptive evolution to produce the mixed sugar cell. The above cell may be constructed using recombinant expression techniques.
e) sampling a single colony isolate
f) subjecting the single colony isolate to adaptive evolution in sequential batch reactors
g) sampling single colony isolates
h) characterizing the single colony isolates for their sugar consumption properties
These steps will be described in details below.

Recombinant Expression

The cell of the invention is a recombinant cell. That is to say, a cell of the invention comprises, or is transformed with or is genetically modified with a nucleotide sequence that does not naturally occur in the cell in question.

Techniques for the recombinant expression of enzymes in a cell, as well as for the additional genetic modifications of a cell of the invention are well known to those skilled in the art. Typically such techniques involve transformation of a cell with nucleic acid construct comprising the relevant sequence. Such methods are, for example, known from standard handbooks, such as Sambrook and Russel (2001) “Molecular Cloning: A Laboratory Manual (3rd edition), Cold Spring Harbor Laboratory, Cold Spring Harbor Laboratory Press, or F. Ausubel et al., eds., “Current protocols in molecular biology”, Green Publishing and Wiley Interscience, New York (1987). Methods for transformation and genetic modification of fungal host cells are known from e.g. EP-A-0635 574, WO 98/46772, WO 99/60102, WO 00/37671, WO90/14423, EP-A-0481008, EP-A-0635574 and U.S. Pat. No. 6,265,186.

Typically, the nucleic acid construct may be a plasmid, for instance a low copy plasmid or a high copy plasmid. The cell according to the present invention may comprise a single or multiple copies of the nucleotide sequence encoding a enzyme, for instance by multiple copies of a nucleotide construct or by use of construct which has multiple copies of the enzyme sequence.

The nucleic acid construct may be maintained episomally and thus comprise a sequence for autonomous replication, such as an autosomal replication sequence sequence. A suitable episomal nucleic acid construct may e.g. be based on the yeast 2μ or pKD1 plasmids (Gleer et al., 1991, Biotechnology 9: 968-975), or the AMA plasmids (Fierro et al., 1995, Curr Genet. 29:482-489). Alternatively, each nucleic acid construct may be integrated in one or more copies into the genome of the cell. Integration into the cell's genome may occur at random by non-homologous recombination but preferably, the nucleic acid construct may be integrated into the cell's genome by homologous recombination as is well known in the art (see e.g. WO90/14423, EP-A-0481008, EP-A-0635 574 and U.S. Pat. No. 6,265,186).

Most episomal or 2μ plasmids are relatively unstable, being lost in approximately 10⁻² or more cells after each generation. Even under conditions of selective growth, only 60% to 95% of the cells retain the episomal plasmid. The copy number of most episomal plasmids ranges from 10-40 per cell of cir⁺ hosts. However, the plasmids are not equally distributed among the cells, and there is a high variance in the copy number per cell in populations. Strains transformed with integrative plasmids are extremely stable, even in the absence of selective pressure. However, plasmid loss can occur at approximately 10⁻³ to 10⁻⁴ frequencies by homologous recombination between tandemly repeated DNA, leading to looping out of the vector sequence. Preferably, the vector design in the case of stable integration is thus, that upon loss of the selection marker genes (which also occurs by intramolecular, homologous recombination) that looping out of the integrated construct is no longer possible. Preferably the genes are thus stably integrated. Stable integration is herein defined as integration into the genome, wherein looping out of the integrated construct is no longer possible. Preferably selection markers are absent. Typically, the enzyme encoding sequence will be operably linked to one or more nucleic acid sequences, capable of providing for or aiding the transcription and/or translation of the enzyme sequence.

The term “operably linked” refers to a juxtaposition wherein the components described are in a relationship permitting them to function in their intended manner. For instance, a promoter or enhancer is operably linked to a coding sequence the said promoter or enhancer affects the transcription of the coding sequence.

As used herein, the term “promoter” refers to a nucleic acid fragment that functions to control the transcription of one or more genes, located upstream with respect to the direction of transcription of the transcription initiation site of the gene, and is structurally identified by the presence of a binding site for DNA-dependent RNA polymerase, transcription initiation sites and any other DNA sequences known to one of skilled in the art. A “constitutive” promoter is a promoter that is active under most environmental and developmental conditions. An “inducible” promoter is a promoter that is active under environmental or developmental regulation.

The promoter that could be used to achieve the expression of a nucleotide sequence coding for an enzyme according to the present invention, may be not native to the nucleotide sequence coding for the enzyme to be expressed, i.e. a promoter that is heterologous to the nucleotide sequence (coding sequence) to which it is operably linked.

The promoter may, however, be homologous, i.e. endogenous, to the host cell. Promotors are widely available and known to the skilled person. Suitable examples of such promoters include e.g. promoters from glycolytic genes, such as the phosphofructokinase (PFK), triose phosphate isomerase (TPI), glyceraldehyde-3-phosphate dehydrogenase (GPD, TDH3 or GAPDH), pyruvate kinase (PYK), phosphoglycerate kinase (PGK) promoters from yeasts or filamentous fungi; more details about such promoters from yeast may be found in (WO 93/03159). Other useful promoters are ribosomal protein encoding gene promoters, the lactase gene promoter (LAC4), alcohol dehydrogenase promoters (ADHI, ADH4, and the like), and the enolase promoter (ENO). Other promoters, both constitutive and inducible, and enhancers or upstream activating sequences will be known to those of skill in the art. The promoters used in the host cells of the invention may be modified, if desired, to affect their control characteristics. Suitable promoters in this context include both constitutive and inducible natural promoters as well as engineered promoters, which are well known to the person skilled in the art. Suitable promoters in eukaryotic host cells may be GAL7, GAL10, or GAL1, CYC1, HIS3, ADH1, PGL, PH05, GAPDH, ADC1, TRP1, URA3, LEU2, ENO1, TPI1, and AOX1. Other suitable promoters include PDC1, GPD1, PGK1, TEF1, and TDH3.

In a cell of the invention, the 3′-end of the nucleotide acid sequence encoding enzyme preferably is operably linked to a transcription terminator sequence. Preferably the terminator sequence is operable in a host cell of choice, such as e.g. the yeast species of choice. In any case the choice of the terminator is not critical; it may e.g. be from any yeast gene, although terminators may sometimes work if from a non-yeast, eukaryotic, gene. Usually a nucleotide sequence encoding the enzyme comprises a terminator. Preferably, such terminators are combined with mutations that prevent nonsense mediated mRNA decay in the host cell of the invention (see for example: Shirley et al., 2002, Genetics 161:1465-1482).

The transcription termination sequence further preferably comprises a polyadenylation signal.

Optionally, a selectable marker may be present in a nucleic acid construct suitable for use in the invention. As used herein, the term “marker” refers to a gene encoding a trait or a phenotype which permits the selection of, or the screening for, a host cell containing the marker. The marker gene may be an antibiotic resistance gene whereby the appropriate antibiotic can be used to select for transformed cells from among cells that are not transformed. Examples of suitable antibiotic resistance markers include e.g. dihydrofolate reductase, hygromycin-B-phosphotransferase, 3′-O-phosphotransferase II (kanamycin, neomycin and G418 resistance). Antibiotic resistance markers may be most convenient for the transformation of polyploid host cells, Also non-antibiotic resistance markers may be used, such as auxotrophic markers (URA3, TRPI, LEU2) or the S. pombe TPI gene (described by Russell P R, 1985, Gene 40: 125-130). In a preferred embodiment the host cells transformed with the nucleic acid constructs are marker gene free. Methods for constructing recombinant marker gene free microbial host cells are disclosed in EP-A-0 635 574 and are based on the use of bidirectional markers such as the A. nidulans amdS (acetamidase) gene or the yeast URA3 and LYS2 genes. Alternatively, a screenable marker such as Green Fluorescent Protein, lacL, luciferase, chloramphenicol acetyltransferase, beta-glucuronidase may be incorporated into the nucleic acid constructs of the invention allowing to screen for transformed cells.

Optional further elements that may be present in the nucleic acid constructs suitable for use in the invention include, but are not limited to, one or more leader sequences, enhancers, integration factors, and/or reporter genes, intron sequences, centromers, telomers and/or matrix attachment (MAR) sequences. The nucleic acid constructs of the invention may further comprise a sequence for autonomous replication, such as an ARS sequence.

The recombination process may thus be executed with known recombination techniques. Various means are known to those skilled in the art for expression and overexpression of enzymes in a cell of the invention. In particular, an enzyme may be overexpressed by increasing the copy number of the gene coding for the enzyme in the host cell, e.g. by integrating additional copies of the gene in the host cell's genome, by expressing the gene from an episomal multicopy expression vector or by introducing a episomal expression vector that comprises multiple copies of the gene.

Alternatively, overexpression of enzymes in the host cells of the invention may be achieved by using a promoter that is not native to the sequence coding for the enzyme to be overexpressed, i.e. a promoter that is heterologous to the coding sequence to which it is operably linked. Although the promoter preferably is heterologous to the coding sequence to which it is operably linked, it is also preferred that the promoter is homologous, i.e. endogenous to the host cell. Preferably the heterologous promoter is capable of producing a higher steady state level of the transcript comprising the coding sequence (or is capable of producing more transcript molecules, i.e. mRNA molecules, per unit of time) than is the promoter that is native to the coding sequence. Suitable promoters in this context include both constitutive and inducible natural promoters as well as engineered promoters.

The coding sequence used for overexpression of the enzymes mentioned above may preferably be homologous to the host cell of the invention. However, coding sequences that are heterologous to the host cell of the invention may be used.

Overexpression of an enzyme, when referring to the production of the enzyme in a genetically modified cell, means that the enzyme is produced at a higher level of specific enzymatic activity as compared to the unmodified host cell under identical conditions. Usually this means that the enzymatically active protein (or proteins in case of multi-subunit enzymes) is produced in greater amounts, or rather at a higher steady state level as compared to the unmodified host cell under identical conditions. Similarly this usually means that the mRNA coding for the enzymatically active protein is produced in greater amounts, or again rather at a higher steady state level as compared to the unmodified host cell under identical conditions. Preferably in a host cell of the invention, an enzyme to be overexpressed is overexpressed by at least a factor of about 1.1, about 1.2, about 1.5, about 2, about 5, about 10 or about 20 as compared to a strain which is genetically identical except for the genetic modification causing the overexpression. It is to be understood that these levels of overexpression may apply to the steady state level of the enzyme's activity, the steady state level of the enzyme's protein as well as to the steady state level of the transcript coding for the enzyme.

### The Adaptive Evolution

The mixed sugar cells are in their preparation subjected to adaptive evolution. A cell of the invention may be adapted to sugar utilisation by selection of mutants, either spontaneous or induced (e.g. by radiation or chemicals), for growth on the desired sugar, preferably as sole carbon source, and more preferably under anaerobic conditions. Selection of mutants may be performed by techniques including serial transfer of cultures as e.g. described by Kuyper et al. (2004, FEMS Yeast Res. 4: 655-664) or by cultivation under selective pressure in a chemostat culture. E.g. in a preferred host cell of the invention at least one of the genetic modifications described above, including modifications obtained by selection of mutants, confer to the host cell the ability to grow on the xylose as carbon source, preferably as sole carbon source, and preferably under anaerobic conditions. Preferably the cell produce essentially no xylitol, e.g. the xylitol produced is below the detection limit or e.g. less than about 5, about 2, about 1, about 0.5, or about 0.3% of the carbon consumed on a molar basis.

Adaptive evolution is also described e.g. in Wisselink H. W. et al, Applied and Environmental Microbiology August 2007, p. 4881-4891

In one embodiment of adaptive evolution a regimen consisting of repeated batch cultivation with repeated cycles of consecutive growth in different media is applied, e.g. three media with different compositions (glucose, xylose, and arabinose; xylose and arabinose. See Wisselink et al. (2009) Applied and Environmental Microbiology, February 2009, p. 907-914.

In one embodiment, the yeast cell BIE252 was adapted in a SBR set-up. The following media were used: (1) mixed sugars medium: 10 g/l glucose, 10 g/l xylose, 7 g/l arabinose, 2 g/l galactose and 1 g/l mannose; (2) arabinose medium: 27 g/l arabinose and 3 g/l xylose and (3) xylose medium: 27 g/l xylose and 3 g/l arabinose. After completion of the batch growth in medium (1), media (2) and (3) were alternated and that sequence of cultivation in media (2) and (3) was repeated for six cycles. Growth in medium (1) was repeated after cycle three to confirm that the culture was still able to utilize the C6-sugars as fast as in the beginning of the SBR cultivation. For each run, the maximum specific growth rate (μₘₐₓ) was estimated from the CO₂ profile in the exponential growth phase.

The Host Cell

The host cell may be any host cell suitable for production of a useful product. A cell of the invention may be any suitable cell, such as a prokaryotic cell, such as a bacterium, or a eukaryotic cell. Typically, the cell will be a eukaryotic cell, for example a yeast or a filamentous fungus.

Yeasts are herein defined as eukaryotic microorganisms and include all species of the subdivision Eumycotina (Alexopoulos, C. J., 1962, In: Introductory Mycology, John Wiley & Sons, Inc., New York) that predominantly grow in unicellular form.

Yeasts may either grow by budding of a unicellular thallus or may grow by fission of the organism. A preferred yeast as a cell of the invention may belong to the genera Saccharomyces, Kluyveromyces, Candida, Pichia, Schizosaccharomyces, Hansenula, Kloeckera, Schwanniomyces or Yarrowia. Preferably the yeast is one capable of anaerobic fermentation, more preferably one capable of anaerobic alcoholic fermentation.

Filamentous fungi are herein defined as eukaryotic microorganisms that include all filamentous forms of the subdivision Eumycotina. These fungi are characterized by a vegetative mycelium composed of chitin, cellulose, and other complex polysaccharides. The filamentous fungi of the suitable for use as a cell of the present invention are morphologically, physiologically, and genetically distinct from yeasts. Filamentous fungal cells may be advantageously used since most fungi do not require sterile conditions for propagation and are insensitive to bacteriophage infections. Vegetative growth by filamentous fungi is by hyphal elongation and carbon catabolism of most filamentous fungi is obligately aerobic. Preferred filamentous fungi as a host cell of the invention may belong to the genus Aspergillus, Trichoderma, Humicola, Acremoniurra, Fusarium or Penicillium. More preferably, the filamentous fungal cell may be a Aspergillus niger, Aspergillus oryzae, a Penicillium chrysogenum, or Rhizopus oryzae cell.

In one embodiment the host cell may be yeast.

Preferably the host is an industrial host, more preferably an industrial yeast. An industrial host and industrial yeast cell may be defined as follows. The living environments of yeast cells in industrial processes are significantly different from that in the laboratory. Industrial yeast cells must be able to perform well under multiple environmental conditions which may vary during the process. Such variations include change in nutrient sources, pH, ethanol concentration, temperature, oxygen concentration, etc., which together have potential impact on the cellular growth and ethanol production of Saccharomyces cerevisiae. Under adverse industrial conditions, the environmental tolerant strains should allow robust growth and production. Industrial yeast strains are generally more robust towards these changes in environmental conditions which may occur in the applications they are used, such as in the baking industry, brewing industry, wine making and the ethanol industry. Examples of industrial yeast (S. cerevisiae) are Ethanol Red® (Fermentis) Fermiol® (DSM) and Thermosacc® (Lallemand).

In an embodiment the host is inhibitor tolerant. Inhibitor tolerant host cells may be selected by screening strains for growth on inhibitors containing materials, such as illustrated in Kadar et al, Appl. Biochem. Biotechnol. (2007), Vol. 136-140, 847-858, wherein an inhibitor tolerant S. cerevisiae strain ATCC 26602 was selected.

Preferably the host cell is industrial and inhibitor tolerant.

araA, araB and araD Genes

A cell of the invention is capable of using arabinose. A cell of the invention is therefore, be capable of converting L-arabinose into L-ribulose and/or xylulose 5-phosphate and/or into a desired fermentation product, for example one of those mentioned herein.

Organisms, for example S. cerevisiae strains, able to produce ethanol from L-arabinose may be produced by modifying a cell introducing the araA (L-arabinose isomerase), araB (L-ribulokinase) and araD (L-ribulose-5-P4-epimerase) genes from a suitable source. Such genes may be introduced into a cell of the invention is order that it is capable of using arabinose. Such an approach is given is described in WO2003/095627. araA, araB and araD genes from Lactobacillus plantanum may be used and are disclosed in WO2008/041840. The araA gene from Bacillus subtilis and the araB and araD genes from Escherichia coli may be used and are disclosed in EP1499708.

PPP-Genes

A cell of the invention may comprise one ore more genetic modifications that increases the flux of the pentose phosphate pathway. In particular, the genetic modification(s) may lead to an increased flux through the non-oxidative part pentose phosphate pathway. A genetic modification that causes an increased flux of the non-oxidative part of the pentose phosphate pathway is herein understood to mean a modification that increases the flux by at least a factor of about 1.1, about 1.2, about 1.5, about 2, about 5, about 10 or about 20 as compared to the flux in a strain which is genetically identical except for the genetic modification causing the increased flux. The flux of the non-oxidative part of the pentose phosphate pathway may be measured by growing the modified host on xylose as sole carbon source, determining the specific xylose consumption rate and subtracting the specific xylitol production rate from the specific xylose consumption rate, if any xylitol is produced. However, the flux of the non-oxidative part of the pentose phosphate pathway is proportional with the growth rate on xylose as sole carbon source, preferably with the anaerobic growth rate on xylose as sole carbon source. There is a linear relation between the growth rate on xylose as sole carbon source (μₘₐₓ) and the flux of the non-oxidative part of the pentose phosphate pathway. The specific xylose consumption rate (Qₛ) is equal to the growth rate (μ) divided by the yield of biomass on sugar (Yₓₛ) because the yield of biomass on sugar is constant (under a given set of conditions: anaerobic, growth medium, pH, genetic background of the strain, etc.; i.e. Qₛ=μ/Yₓₛ). Therefore the increased flux of the non-oxidative part of the pentose phosphate pathway may be deduced from the increase in maximum growth rate under these conditions unless transport (uptake is limiting).

One or more genetic modifications that increase the flux of the pentose phosphate pathway may be introduced in the host cell in various ways. These including e.g. achieving higher steady state activity levels of xylulose kinase and/or one or more of the enzymes of the non-oxidative part pentose phosphate pathway and/or a reduced steady state level of unspecific aldose reductase activity. These changes in steady state activity levels may be effected by selection of mutants (spontaneous or induced by chemicals or radiation) and/or by recombinant DNA technology e.g. by overexpression or inactivation, respectively, of genes encoding the enzymes or factors regulating these genes.

In a preferred host cell, the genetic modification comprises overexpression of at least one enzyme of the (non-oxidative part) pentose phosphate pathway. Preferably the enzyme is selected from the group consisting of the enzymes encoding for ribulose-5-phosphate isomerase, ribulose-5-phosphate epimerase, transketolase and transaldolase. Various combinations of enzymes of the (non-oxidative part) pentose phosphate pathway may be overexpressed. E.g. the enzymes that are overexpressed may be at least the enzymes ribulose-5-phosphate isomerase and ribulose-5-phosphate epimerase; or at least the enzymes ribulose-5-phosphate isomerase and transketolase; or at least the enzymes ribulose-5-phosphate isomerase and transaldolase; or at least the enzymes ribulose-5-phosphate epimerase and transketolase; or at least the enzymes ribulose-5-phosphate epimerase and transaldolase; or at least the enzymes transketolase and transaldolase; or at least the enzymes ribulose-5-phosphate epimerase, transketolase and transaldolase; or at least the enzymes ribulose-5-phosphate isomerase, transketolase and transaldolase; or at least the enzymes ribulose-5-phosphate isomerase, ribulose-5-phosphate epimerase, and transaldolase; or at least the enzymes ribulose-5-phosphate isomerase, ribulose-5-phosphate epimerase, and transketolase. In one embodiment of the invention each of the enzymes ribulose-5-phosphate isomerase, ribulose-5-phosphate epimerase, transketolase and transaldolase are overexpressed in the host cell. More preferred is a host cell in which the genetic modification comprises at least overexpression of both the enzymes transketolase and transaldolase as such a host cell is already capable of anaerobic growth on xylose. In fact, under some conditions host cells overexpressing only the transketolase and the transaldolase already have the same anaerobic growth rate on xylose as do host cells that overexpress all four of the enzymes, i.e. the ribulose-5-phosphate isomerase, ribulose-5-phosphate epimerase, transketolase and transaldolase. Moreover, host cells overexpressing both of the enzymes ribulose-5-phosphate isomerase and ribulose-5-phosphate epimerase are preferred over host cells overexpressing only the isomerase or only the epimerase as overexpression of only one of these enzymes may produce metabolic imbalances.

The enzyme “ribulose 5-phosphate epimerase” (EC 5.1.3.1) is herein defined as an enzyme that catalyses the epimerisation of D-xylulose 5-phosphate into D-ribulose 5-phosphate and vice versa. The enzyme is also known as phosphoribulose epimerase; erythrose-4-phosphate isomerase; phosphoketopentose 3-epimerase; xylulose phosphate 3-epimerase; phosphoketopentose epimerase; ribulose 5-phosphate 3-epimerase; D-ribulose phosphate-3-epimerase; D-ribulose 5-phosphate epimerase; D-ribulose-5-P 3-epimerase; D-xylulose-5-phosphate 3-epimerase; pentose-5-phosphate 3-epimerase; or D-ribulose-5-phosphate 3-epimerase. A ribulose 5-phosphate epimerase may be further defined by its amino acid sequence. Likewise a ribulose 5-phosphate epimerase may be defined by a nucleotide sequence encoding the enzyme as well as by a nucleotide sequence hybridising to a reference nucleotide sequence encoding a ribulose 5-phosphate epimerase. The nucleotide sequence encoding for ribulose 5-phosphate epimerase is herein designated RPE1.

The enzyme “ribulose 5-phosphate isomerase” (EC 5.3.1.6) is herein defined as an enzyme that catalyses direct isomerisation of D-ribose 5-phosphate into D-ribulose 5-phosphate and vice versa. The enzyme is also known as phosphopentosisomerase; phosphoriboisomerase; ribose phosphate isomerase; 5-phosphoribose isomerase; D-ribose 5-phosphate isomerase; D-ribose-5-phosphate ketol-isomerase; or D-ribose-5-phosphate aldose-ketose-isomerase. A ribulose 5-phosphate isomerase may be further defined by its amino acid sequence. Likewise a ribulose 5-phosphate isomerase may be defined by a nucleotide sequence encoding the enzyme as well as by a nucleotide sequence hybridising to a reference nucleotide sequence encoding a ribulose 5-phosphate isomerase. The nucleotide sequence encoding for ribulose 5-phosphate isomerase is herein designated RPI1.

The enzyme “transketolase” (EC 2.2.1.1) is herein defined as an enzyme that catalyses the reaction: D-ribose 5-phosphate+D-xylulose 5-phosphate<->sedoheptulose 7-phosphate+D-glyceraldehyde 3-phosphate and vice versa. The enzyme is also known as glycolaldehydetransferase or sedoheptulose-7-phosphate:D-glyceraldehyde-3-phosphate glycolaldehydetransferase. A transketolase may be further defined by its amino acid. Likewise a transketolase may be defined by a nucleotide sequence encoding the enzyme as well as by a nucleotide sequence hybridising to a reference nucleotide sequence encoding a transketolase. The nucleotide sequence encoding for transketolase is herein designated TKL1.

The enzyme “transaldolase” (EC 2.2.1.2) is herein defined as an enzyme that catalyses the reaction: sedoheptulose 7-phosphate+D-glyceraldehyde 3-phosphate<->D-erythrose 4-phosphate+D-fructose 6-phosphate and vice versa. The enzyme is also known as dihydroxyacetonetransferase; dihydroxyacetone synthase; formaldehyde transketolase; or sedoheptulose-7-phosphate:D-glyceraldehyde-3-phosphate glyceronetransferase. A transaldolase may be further defined by its amino acid sequence. Likewise a transaldolase may be defined by a nucleotide sequence encoding the enzyme as well as by a nucleotide sequence hybridising to a reference nucleotide sequence encoding a transaldolase. The nucleotide sequence encoding for transketolase from is herein designated TAL1.

### Xylose Isomerase Gene

The presence of the nucleotide sequence encoding a xylose isomerase confers on the cell the ability to isomerise xylose to xylulose. According to the invention, two to fifteen copies of one or more xylose isomerase gene are introduced into the host cell.

In one embodiment, the two to fifteen copies of one or more xylose isomerase gene are introduced into the host cell.

A “xylose isomerase” (EC 5.3.1.5) is herein defined as an enzyme that catalyses the direct isomerisation of D-xylose into D-xylulose and/or vice versa. The enzyme is also known as a D-xylose ketoisomerase. A xylose isomerase herein may also be capable of catalysing the conversion between D-glucose and D-fructose (and accordingly may therefore be referred to as a glucose isomerase). A xylose isomerase herein may require a bivalent cation, such as magnesium, manganese or cobalt as a cofactor.

Accordingly, a cell of the invention is capable of isomerising xylose to xylulose. The ability of isomerising xylose to xylulose is conferred on the host cell by transformation of the host cell with a nucleic acid construct comprising a nucleotide sequence encoding a defined xylose isomerase. A cell of the invention isomerises xylose into xylulose by the direct isomerisation of xylose to xylulose. This is understood to mean that xylose is isomerised into xylulose in a single reaction catalysed by a xylose isomerase, as opposed to two step conversion of xylose into xylulose via a xylitol intermediate as catalysed by xylose reductase and xylitol dehydrogenase, respectively.

A unit (U) of xylose isomerase activity may herein be defined as the amount of enzyme producing 1 nmol of xylulose per minute, under conditions as described by Kuyper et al. (2003, FEMS Yeast Res. 4: 69-78). The Xylose isomerise gene may have various origin, such as for example Pyromyces sp. as disclosed in WO2006/009434. Other suitable origins are Bacteroides, in particular Bacteroides unifomis as described in PCT/EP2009/52623, Bacillus, in particular Bacillus stearothermophilus as described in PCT/EP2009/052625, Thermotoga, in particular Thermotoga maritima, as described in PCT/EP2009/052621 and Clostridium, in particular Clostridium cellulolyticum as described in PCT/EP2009/052620.

XKS1 Gene

A cell of the invention may comprise one or more genetic modifications that increase the specific xylulose kinase activity. Preferably the genetic modification or modifications causes overexpression of a xylulose kinase, e.g. by overexpression of a nucleotide sequence encoding a xylulose kinase. The gene encoding the xylulose kinase may be endogenous to the host cell or may be a xylulose kinase that is heterologous to the host cell. A nucleotide sequence used for overexpression of xylulose kinase in the host cell of the invention is a nucleotide sequence encoding a polypeptide with xylulose kinase activity.

The enzyme “xylulose kinase” (EC 2.7.1.17) is herein defined as an enzyme that catalyses the reaction ATP+D-xylulose=ADP+D-xylulose 5-phosphate. The enzyme is also known as a phosphorylating xylulokinase, D-xylulokinase or ATP:D-xylulose 5-phosphotransf erase. A xylulose kinase of the invention may be further defined by its amino acid sequence. Likewise a xylulose kinase may be defined by a nucleotide sequence encoding the enzyme as well as by a nucleotide sequence hybridising to a reference nucleotide sequence encoding a xylulose kinase.

In a cell of the invention, a genetic modification or modifications that increase(s) the specific xylulose kinase activity may be combined with any of the modifications increasing the flux of the pentose phosphate pathway as described above. This is not, however, essential.

Thus, a host cell of the invention may comprise only a genetic modification or modifications that increase the specific xylulose kinase activity. The various means available in the art for achieving and analysing overexpression of a xylulose kinase in the host cells of the invention are the same as described above for enzymes of the pentose phosphate pathway. Preferably in the host cells of the invention, a xylulose kinase to be overexpressed is overexpressed by at least a factor of about 1.1, about 1.2, about 1.5, about 2, about 5, about 10 or about 20 as compared to a strain which is genetically identical except for the genetic modification(s) causing the overexpression. It is to be understood that these levels of overexpression may apply to the steady state level of the enzyme's activity, the steady state level of the enzyme's protein as well as to the steady state level of the transcript coding for the enzyme.

Aldose Reductase (GRE3) Gene Deletion

A cell of the invention may comprise one or more genetic modifications that reduce unspecific aldose reductase activity in the host cell. Preferably, unspecific aldose reductase activity is reduced in the host cell by one or more genetic modifications that reduce the expression of or inactivates a gene encoding an unspecific aldose reductase. Preferably, the genetic modification(s) reduce or inactivate the expression of each endogenous copy of a gene encoding an unspecific aldose reductase in the host cell (herein called GRE3 deletion). Host cells may comprise multiple copies of genes encoding unspecific aldose reductases as a result of di-, poly- or aneu-ploidy, and/or the host cell may contain several different (iso)enzymes with aldose reductase activity that differ in amino acid sequence and that are each encoded by a different gene. Also in such instances preferably the expression of each gene that encodes an unspecific aldose reductase is reduced or inactivated. Preferably, the gene is inactivated by deletion of at least part of the gene or by disruption of the gene, whereby in this context the term gene also includes any non-coding sequence up- or down-stream of the coding sequence, the (partial) deletion or inactivation of which results in a reduction of expression of unspecific aldose reductase activity in the host cell.

A nucleotide sequence encoding an aldose reductase whose activity is to be reduced in the host cell of the invention is a nucleotide sequence encoding a polypeptide with aldose reductase activity.

Thus, a host cell of the invention comprising only a genetic modification or modifications that reduce(s) unspecific aldose reductase activity in the host cell is specifically included in the invention.

The enzyme “aldose reductase” (EC 1.1.1.21) is herein defined as any enzyme that is capable of reducing xylose or xylulose to xylitol. In the context of the present invention an aldose reductase may be any unspecific aldose reductase that is native (endogenous) to a host cell of the invention and that is capable of reducing xylose or xylulose to xylitol. Unspecific aldose reductases catalyse the reaction:

aldose+NAD(P)H+H⁺ [symbol] alditol+NAD(P)⁺

The enzyme has a wide specificity and is also known as aldose reductase; polyol dehydrogenase (NADP⁺); alditol:NADP oxidoreductase; alditol:NADP⁺1-oxidoreductase; NADPH-aldopentose reductase; or NADPH-aldose reductase.

A particular example of such an unspecific aldose reductase that is endogenous to S. cerevisiae and that is encoded by the GRE3 gene (Traff et al., 2001, Appl. Environ. Microbiol. 67: 5668-74). Thus, an aldose reductase of the invention may be further defined by its amino acid sequence. Likewise an aldose reductase may be defined by the nucleotide sequences encoding the enzyme as well as by a nucleotide sequence hybridising to a reference nucleotide sequence encoding an aldose reductase.

Bioproducts Production

Over the years suggestions have been made for the introduction of various organisms for the production of bio-ethanol from crop sugars. In practice, however, all major bio-ethanol production processes have continued to use the yeasts of the genus Saccharomyces as ethanol producer. This is due to the many attractive features of Saccharomyces species for industrial processes, i.e., a high acid-, ethanol- and osmo-tolerance, capability of anaerobic growth, and of course its high alcoholic fermentative capacity. Preferred yeast species as host cells include S. cerevisiae, S. bulderi, S. barnetti, S. exiguus, S. uvarum, S. diastaticus, K. lactis, K. marxianus or K. fragilis.

A cell of the invention may be able to convert plant biomass, celluloses, hemicelluloses, pectins, rhamnose, galactose, frucose, maltose, maltodextrines, ribose, ribulose, or starch, starch derivatives, sucrose, lactose and glycerol, for example into fermentable sugars. Accordingly, a cell of the invention may express one or more enzymes such as a cellulase (an endocellulase or an exocellulase), a hemicellulase (an endo- or exo-xylanase or arabinase) necessary for the conversion of cellulose into glucose monomers and hemicellulose into xylose and arabinose monomers, a pectinase able to convert pectins into glucuronic acid and galacturonic acid or an amylase to convert starch into glucose monomers.

The cell further preferably comprises those enzymatic activities required for conversion of pyruvate to a desired fermentation product, such as ethanol, butanol, lactic acid, 3-hydroxy-propionic acid, acrylic acid, acetic acid, succinic acid, citric acid, fumaric acid, malic acid, itaconic acid, an amino acid, 1,3-propane-diol, ethylene, glycerol, a β-lactam antibiotic or a cephalosporin.

A preferred cell of the invention is a cell that is naturally capable of alcoholic fermentation, preferably, anaerobic alcoholic fermentation. A cell of the invention preferably has a high tolerance to ethanol, a high tolerance to low pH (i.e. capable of growth at a pH lower than about 5, about 4, about 3, or about 2.5) and towards organic acids like lactic acid, acetic acid or formic acid and/or sugar degradation products such as furfural and hydroxy-methylfurfural and/or a high tolerance to elevated temperatures.

Any of the above characteristics or activities of a cell of the invention may be naturally present in the cell or may be introduced or modified by genetic modification.

A cell of the invention may be a cell suitable for the production of ethanol. A cell of the invention may, however, be suitable for the production of fermentation products other than ethanol. Such non-ethanolic fermentation products include in principle any bulk or fine chemical that is producible by a eukaryotic microorganism such as a yeast or a filamentous fungus.

Such fermentation products may be, for example, butanol, lactic acid, 3-hydroxy-propionic acid, acrylic acid, acetic acid, succinic acid, citric acid, malic acid, fumaric acid, itaconic acid, an amino acid, 1,3-propane-diol, ethylene, glycerol, a β-lactam antibiotic or a cephalosporin. A preferred cell of the invention for production of non-ethanolic fermentation products is a host cell that contains a genetic modification that results in decreased alcohol dehydrogenase activity.

In a further aspect the invention relates to fermentation processes in which the cells of the invention are used for the fermentation of a carbon source comprising a source of xylose, such as xylose. In addition to a source of xylose the carbon source in the fermentation medium may also comprise a source of glucose. The source of xylose or glucose may be xylose or glucose as such or may be any carbohydrate oligo- or polymer comprising xylose or glucose units, such as e.g. lignocellulose, xylans, cellulose, starch and the like. For release of xylose or glucose units from such carbohydrates, appropriate carbohydrases (such as xylanases, glucanases, amylases and the like) may be added to the fermentation medium or may be produced by the cell. In the latter case the cell may be genetically engineered to produce and excrete such carbohydrases. An additional advantage of using oligo- or polymeric sources of glucose is that it enables to maintain a low(er) concentration of free glucose during the fermentation, e.g. by using rate-limiting amounts of the carbohydrases. This, in turn, will prevent repression of systems required for metabolism and transport of non-glucose sugars such as xylose.

In a preferred process the cell ferments both the xylose and glucose, preferably simultaneously in which case preferably a cell is used which is insensitive to glucose repression to prevent diauxic growth. In addition to a source of xylose (and glucose) as carbon source, the fermentation medium will further comprise the appropriate ingredient required for growth of the cell. Compositions of fermentation media for growth of microorganisms such as yeasts are well known in the art. The fermentation process is a process for the production of a fermentation product such as e.g. ethanol, butanol, lactic acid, 3-hydroxy-propionic acid, acrylic acid, acetic acid, succinic acid, citric acid, malic acid, fumaric acid, itaconic acid, an amino acid, 1,3-propane-diol, ethylene, glycerol, a β-lactam antibiotic, such as Penicillin G or Penicillin V and fermentative derivatives thereof, and a cephalosporin.

Bioproducts Production

Over the years suggestions have been made for the introduction of various organisms for the production of bio-ethanol from crop sugars. In practice, however, all major bio-ethanol production processes have continued to use the yeasts of the genus Saccharomyces as ethanol producer. This is due to the many attractive features of Saccharomyces species for industrial processes, i.e., a high acid-, ethanol- and osmo-tolerance, capability of anaerobic growth, and of course its high alcoholic fermentative capacity. Preferred yeast species as host cells include S. cerevisiae, S. bulderi, S. barnetti, S. exiguus, S. uvarum, S. diastaticus, K. lactis, K. marxianus or K. fragilis.

A mixed sugar cell may be a cell suitable for the production of ethanol. A mixed sugar cell may, however, be suitable for the production of fermentation products other than ethanol. Such non-ethanolic fermentation products include in principle any bulk or fine chemical that is producible by a eukaryotic microorganism such as a yeast or a filamentous fungus.

A mixed sugar cell may be used for production of non-ethanolic fermentation products is a host cell that contains a genetic modification that results in decreased alcohol dehydrogenase activity.

In an embodiment the mixed sugar cell may be used in a process wherein sugars originating from lignocellulose are converted into ethanol.

Lignocellulose

Lignocellulose, which may be considered as a potential renewable feedstock, generally comprises the polysaccharides cellulose (glucans) and hemicelluloses (xylans, heteroxylans and xyloglucans). In addition, some hemicellulose may be present as glucomannans, for example in wood-derived feedstocks. The enzymatic hydrolysis of these polysaccharides to soluble sugars, including both monomers and multimers, for example glucose, cellobiose, xylose, arabinose, galactose, fructose, mannose, rhamnose, ribose, galacturonic acid, glucoronic acid and other hexoses and pentoses occurs under the action of different enzymes acting in concert.

In addition, pectins and other pectic substances such as arabinans may make up considerably proportion of the dry mass of typically cell walls from non-woody plant tissues (about a quarter to half of dry mass may be pectins).

Pretreatment

Before enzymatic treatment, the lignocellulosic material may be pretreated. The pretreatment may comprise exposing the lignocellulosic material to an acid, a base, a solvent, heat, a peroxide, ozone, mechanical shredding, grinding, milling or rapid depressurization, or a combination of any two or more thereof. This chemical pretreatment is often combined with heat-pretreatment, e.g. between 150-220° C. for 1 to 30 minutes.

Enzymatic Hydrolysis

The pretreated material is commonly subjected to enzymatic hydrolysis to release sugars that may be fermented according to the invention. This may be executed with conventional methods, e.g. contacting with cellulases, for instance cellobiohydrolase(s), endoglucanase(s), beta-glucosidase(s) and optionally other enzymes. The conversion with the cellulases may be executed at ambient temperatures or at higher temperatures, at a reaction time to release sufficient amounts of sugar(s). The result of the enzymatic hydrolysis is hydrolysis product comprising C5/C6 sugars, herein designated as the sugar composition.

Fermentation

The fermentation process may be an aerobic or an anaerobic fermentation process. An anaerobic fermentation process is herein defined as a fermentation process run in the absence of oxygen or in which substantially no oxygen is consumed, preferably less than about 5, about 2.5 or about 1 mmol/L/h, more preferably 0 mmol/L/h is consumed (i.e. oxygen consumption is not detectable), and wherein organic molecules serve as both electron donor and electron acceptors. In the absence of oxygen, NADH produced in glycolysis and biomass formation, cannot be oxidised by oxidative phosphorylation. To solve this problem many microorganisms use pyruvate or one of its derivatives as an electron and hydrogen acceptor thereby regenerating NAD⁺.

Thus, in a preferred anaerobic fermentation process pyruvate is used as an electron (and hydrogen acceptor) and is reduced to fermentation products such as ethanol, butanol, lactic acid, 3-hydroxy-propionic acid, acrylic acid, acetic acid, succinic acid, citric acid, malic acid, fumaric acid, an amino acid, 1,3-propane-diol, ethylene, glycerol, a β-lactam antibiotic and a cephalosporin.

The fermentation process is preferably run at a temperature that is optimal for the cell. Thus, for most yeasts or fungal host cells, the fermentation process is performed at a temperature which is less than about 42° C., preferably less than about 38° C. For yeast or filamentous fungal host cells, the fermentation process is preferably performed at a temperature which is lower than about 35, about 33, about 30 or about 28° C. and at a temperature which is higher than about 20, about 22, or about 25° C.

The ethanol yield on xylose and/or glucose in the process preferably is at least about 50, about 60, about 70, about 80, about 90, about 95 or about 98%. The ethanol yield is herein defined as a percentage of the theoretical maximum yield.

The invention also relates to a process for producing a fermentation product.,

The fermentation processes may be carried out in batch, fed-batch or continuous mode. A separate hydrolysis and fermentation (SHF) process or a simultaneous saccharification and fermentation (SSF) process may also be applied. A combination of these fermentation process modes may also be possible for optimal productivity.

The fermentation process according to the present invention may be run under aerobic and anaerobic conditions. Preferably, the process is carried out under micro-aerophilic or oxygen limited conditions.

An anaerobic fermentation process is herein defined as a fermentation process run in the absence of oxygen or in which substantially no oxygen is consumed, preferably less than about 5, about 2.5 or about 1 mmol/L/h, and wherein organic molecules serve as both electron donor and electron acceptors.

An oxygen-limited fermentation process is a process in which the oxygen consumption is limited by the oxygen transfer from the gas to the liquid. The degree of oxygen limitation is determined by the amount and composition of the in going gasflow as well as the actual mixing/mass transfer properties of the fermentation equipment used. Preferably, in a process under oxygen-limited conditions, the rate of oxygen consumption is at least about 5.5, more preferably at least about 6, such as at least 7 mmol/L/h. A process of the invention comprises recovery of the fermentation product.

Fermentation Product

The fermentation product of the invention may be any useful product. In one embodiment, it is a product selected from the group consisting of ethanol, n-butanol, isobutanol, lactic acid, 3-hydroxy-propionic acid, acrylic acid, acetic acid, succinic acid, fumaric acid, malic acid, itaconic acid, maleic acid, citric acid, adipic acid, an amino acid, such as lysine, methionine, tryptophan, threonine, and aspartic acid, 1,3-propane-diol, ethylene, glycerol, a β-lactam antibiotic and a cephalosporin, vitamins, pharmaceuticals, animal feed supplements, specialty chemicals, chemical feedstocks, plastics, solvents, fuels, including biofuels and biogas or organic polymers, and an industrial enzyme, such as a protease, a cellulase, an amylase, a glucanase, a lactase, a lipase, a lyase, an oxidoreductases, a transferase or a xylanase. For example the fermentation products may be produced by cells according to the invention, following additionally prior art cell preparation methods and fermentation processes, which examples however should herein not be construed as limiting. For example. n-butanol may be produced by cells as described in WO2008121701 or WO2008086124; lactic acid as described in US2011053231 or US2010137551; 3-hydroxy-propionic acid as described in WO2010010291; acrylic acid as described in WO2009153047. An overview of all kind of fermentation products is and how they can be preprared in yeast is given in Romanos, Mass., et al, “Foreign Gene Expression in Yeast: a Review”, yeast vol. 8: 423-488 (1992), see e.g. table 7. The production of glycerol, 1,3 propane diol, organic acids, and vitamin C (table 2) is described in Nevoigt, E., Microbiol. Mol. Biol. Rev. 72(3) 379-412 (2008). Giddijala, L., et al, BMC Biotechnology 8(29) (2008) describes production of beta-lactams in yeast.

Recovery of the Fermentation Product

For the recovery of the fermenation product existing technologies are used. For different fermentation products different recovery processes are appropriate. Existing methods of recovering ethanol from aqueous mixtures commonly use fractionation and adsorption techniques. For example, a beer still can be used to process a fermented product, which contains ethanol in an aqueous mixture, to produce an enriched ethanol-containing mixture that is then subjected to fractionation (e.g., fractional distillation or other like techniques). Next, the fractions containing the highest concentrations of ethanol can be passed through an adsorber to remove most, if not all, of the remaining water from the ethanol.

The following examples illustrate the invention:

### EXAMPLES

Unless indicated otherwise, the methods described in here are standard biochemical techniques. Examples of suitable general methodology textbooks include Sambrook et al., Molecular Cloning, a Laboratory Manual (1989) and Ausubel et al., Current Protocols in Molecular Biology (1995), John Wiley & Sons, Inc.

Medium Composition

Growth experiments: Saccharomyces cerevisiae strains are grown on medium having the following composition: 0.67% (w/v) yeast nitrogen base or synthetic medium (Verduyn et al., Yeast 8:501-517, 1992) and glucose, arabinose, mannose, galactose or xylose, or a combination of these substrates, at varying concentrations (see examples for specific details; concentrations in % weight over volume (w/v)). For agar plates the medium is supplemented with 2% (w/v) bacteriological agar.

Ethanol Production

Pre-cultures were prepared by inoculating 25 ml Verduyn-medium (Verduyn et al., Yeast 8:501-517, 1992) supplemented with 2% glucose in a 100 ml shake flask with a frozen stock culture or a single colony from an agar plate. After incubation at 30° C. in an orbital shaker (280 rpm) for approximately 24 hours, the culture was harvested and used for determination of CO₂ evolution and ethanol production experiments.

Cultivations for ethanol production were performed at 30° C. in 100 ml synthetic model medium (Verduyn-medium (Verduyn et al., Yeast 8:501-517, 1992) supplemented with 5% glucose, 5% xylose, 3.5% arabinose, 1% galactose and 0.5% mannose) in the BAM (Biological Activity Monitor, Halotec, the Netherlands). The pH of the medium was adjusted to 4.2 with 2 M NaOH/H₂SO4 prior to sterilisation. The synthetic medium for anaerobic cultivation was supplemented with 0.01 g l⁻¹ ergosterol and 0.42 g l⁻¹ Tween 80 dissolved in ethanol (Andreasen and Stier. J. Cell Physiol. 41:23-36, 1953; and Andreasen and Stier. J. Cell Physiol. 43:271-281, 1954). The medium was inoculated at an initial OD600 of approximately 2. Cultures were stirred by a magnetic stirrer. Anaerobic conditions developed rapidly during fermentation as the culture was not aerated. CO₂ production was monitored constantly. Sugar conversion and product formation (ethanol, glycerol) was analyzed by NMR. Growth was monitored by following optical density of the culture at 600 nm on a LKB Ultrospec K spectrophotometer.

Transformation of S. cerevisiae

Transformation of S. cerevisiae was done as described by Gietz and Woods (2002; Transformation of the yeast by the LiAc/SS carrier DNA/PEG method. Methods in Enzymology 350: 87-96).

Colony PCR

A single colony isolate was picked with a plastic toothpick and resuspended in 50 μl milliQ water. The sample was incubated for 10 minutes at 99° C. 5 μl of the incubated sample was used as a template for the PCR reaction, using Phusion® DNA polymerase (Finnzymes) according to the instructions provided by the supplier.

PCR Reaction Conditions:

| | | | |
|---|---|---|---|
| step 1 | 3′ | 98° C. | |
| step 2 | 10″ | 98° C. | |
| step 3 | 15″ | 58° C. | repeat step 2 to 4 for 30 cycles |
| step 4 | 30″ | 72° C. | |
| step 5 | 4′ | 72° C. | |
| step 6 | 30″ | 20° C. | |

Chromosomal DNA Isolation

Yeast cells were grown in YEP-medium containing 2% glucose, in a rotary shaker (overnight, at 30° C. and 280 rpm). 1.5 ml of these cultures were transferred to an Eppendorf tube and centrifuged for 1 minute at maximum speed. The supernatant was decanted and the pellet was resuspended in 200 μl of YCPS (0.1% SB3-14 (Sigma Aldrich, the Netherlands) in 10 mM Tris.HCl pH 7.5; 1 mM EDTA) and 1 μl RNase (20 mg/ml RNase A from bovine pancreas, Sigma, the Netherlands). The cell suspension was incubated for 10 minutes at 65° C. The suspension was centrifuged in an Eppendorf centrifuge for 1 minute at 7000 rpm. The supernatant was discarded. The pellet was carefully dissolved in 200 μl CLS (25 mM EDTA, 2% SDS) and 1 μl RNase A. After incubation at 65° C. for 10 minutes, the suspension was cooled on ice. After addition of 70 μl PPS (10M ammonium acetate) the solutions were thoroughly mixed on a Vortex mixer. After centrifugation (5 minutes in Eppendorf centrifuge at maximum speed), the supernatant was mixed with 200 μl ice-cold isopropanol. The DNA readily precipitated and was pelleted by centrifugation (5 minutes, maximum speed). The pellet was washed with 400 μl ice-cold 70% ethanol. The pellet was dried at room temperature and dissolved in 50 μl TE (10 mM Tris.HCl pH7.5, 1 mM EDTA).

Yeast Application Test on Real Hydrolysates

Dilute acid pretreated samples of corn stover were enzymatically hydrolyzed by using an experimental broad spectrum cellulase preparation at 60° C. for 3 days (72 hours). The pH at the start of the hydrolysis was 5.0. The dry matter content at the start of the hydrolysis was 10 and 20% w/w. After hydrolysis (72 hrs), the samples were allowed to be cooled to room temperature. The pH was adjusted to 5.5 using 10% NaOH. Subsequently, 1 milliliter of a 200 gram per liter (NH4)2SO4 and 1 milliliter of 100 gram per liter KH2PO4 was added. Finally, yeast samples were added corresponding to a yeast dry matter content of 1 or 2 gram yeast per kilogram hydrolysate at 10 or 20% w/w, respectively. The CO₂ evolution in time was followed using the AFM (Alcohol Fermentation Monitor; HaloteC Instruments BV, Veenendaal, the Netherlands). Experiments were performed in at least triplicate, for 72 hours at 33° C. One of these is sampled at regular intervals in order to be able to analyze ethanol formation and residual sugar concentrations. These data can be used to calculate fermentation yields. The broth of the other two experiments is not sampled. Instead, at the end of the fermentation the broth is distilled using a Buchi K-355 distillation unit at 45% steam for 15 minutes. The alcohol produced is being determined using an Anton Paar DMA 5000 density meter (Anton Paar Benelux BVBA, Dongen, the Netherlands).

### Example 1

### Growth Rate Improvements on Xylose and Arabinose in Sequential Batch Reactor (SBR) Cultivations

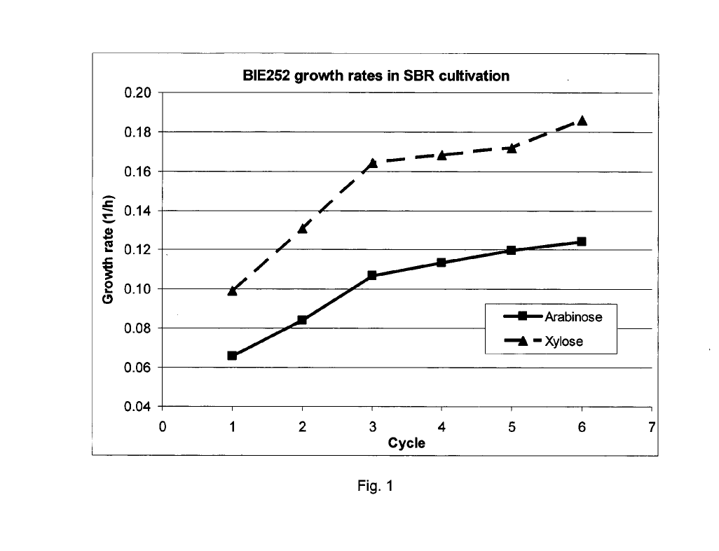

Strain S. cerevisiae BIE252 was grown in a Sequential Batch Reactor cultivation system according to a modified protocol described in WO 2009/112472 in order to improve the growth rates on C5-sugars. Anaerobic cultivation was carried out at 32° C. in 5-L laboratory fermentors with a working volume of 2-L. The pH was maintained at 4.0 by automatic addition of 2 M KOH. The cultures were stirred at 100 rpm and sparged with 0.01 vvm air, while 2 nL/min N₂ was used in the headspace as carrier gas for the MS off-gas measurements. The cultures were performed in media containing different C6- and C5-sugars composition. When the C-source was fully depleted, as indicated by the CO₂ level, a new cycle of batch cultivation was initiated by replacement of ˜95-99% of the culture with fresh synthetic medium containing the appropriate C-source. The following media were used: (1) mixed sugars medium: 10 g/l glucose, 10 g/l xylose, 7 g/l arabinose, 2 g/l galactose and 1 g/l mannose; (2) arabinose medium: 27 g/l arabinose and 3 g/l xylose and (3) xylose medium: 27 g/l xylose and 3 g/l arabinose. After completion of the batch growth in medium (1), media (2) and (3) were alternated and that sequence of cultivation in media (2) and (3) was repeated for six cycles. Growth in medium (1) was repeated after cycle three to confirm that the culture was still able to utilize the C6-sugars as fast as in the beginning of the SBR cultivation. For each run, the maximum specific growth rate (μₘₐₓ) was estimated from the CO₂ profile in the exponential growth phase. After six cycles (90-100 generations) of SBR cultivation both the growth rates of strain S. cerevisiae BIE252 on xylose and arabinose were almost doubled, as depicted in FIG. 1. The growth rate on xylose was increased from 0.1 h⁻¹ to 0.19 h⁻¹, while the growth rate on arabinose from 0.066 h⁻¹ to 0.12 h⁻¹. After the last cycle of evolutionary engineering in the SBR system, a sample was taken for single colony isolation.

### Example 2

### Single Colony Isolation

The following approach was executed in order to select a strain which has gained improved growth on xylose and arabinose as a sole carbon source without losing its ability to utilize C6-sugars (glucose and galactose). Initially, a sample was taken from the fermentor after the last cycle of evolutionary engineering in the SBR cultivation system. The broth sample (SBR culture) was streaked on YEPD-agar and incubated for 48 hours at 30° C. Nine single colony isolates of the SBR culture were re-streaked on YEPD-agar and incubated for 48 hours at 30° C. Subsequently, a pre-culture was done for each single colony on YEP liquid medium supplemented with 2% glucose. The nine cultures were incubated overnight at 30° C. and 280 rpm. All nine cultures were tested for their performance in the BAM (Biological Activity Monitor) system (Halotec, Veenendaal, the Netherlands).

### Example 3

### Performance Test in BAM

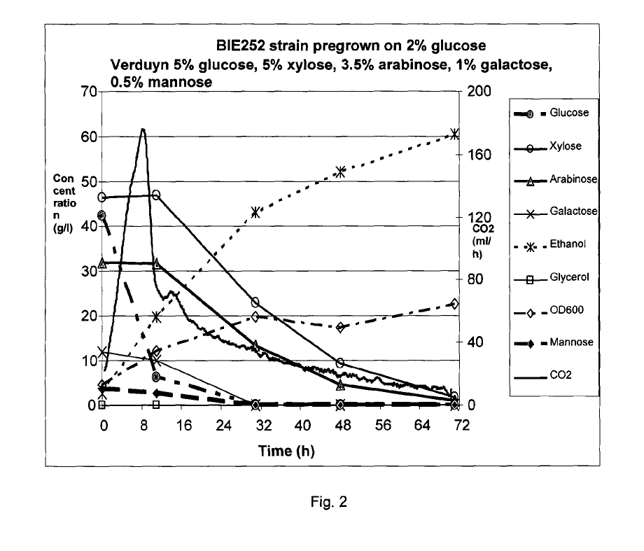

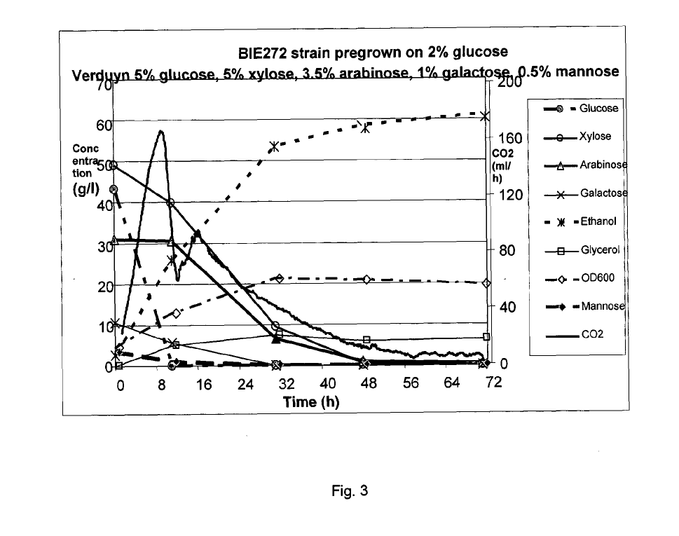

In order to test the performance of the nine single colony isolates, the strains were inoculated in Verduyn medium, supplemented with 2% glucose. As control, strain S. cerevisiae BIE252, the original strain before adaptive evolution in the SBR cultivation system, was included. After overnight incubation at 30° C. and 280 rpm in a rotary shaker, cells were harvested by centrifugation and cultivations for CO2 production were performed at 33° C. and pH 4.2 in 100 ml Verduyn medium supplemented with 50 g/l glucose, 50 g/l xylose, 35 g/l arabinose, 10 g/l galactose and 5 g/l mannose in the BAM. The CO2 production was constantly monitored at intervals, and samples were taken for analysis (optical density at 600 nm using a spectrophotometer; ethanol, glycerol and residual sugars by NMR). Among the nine single colony isolates, colony number five, designated as strain BIE272, performed significantly better than strain BIE252. The results of the BAM experiment of strains BIE252 and BIE272 are shown in FIGS. 2 and 3, respectively. The performance of strain BIE252 shows that glucose was consumed readily after the start of the fermentation experiment (FIG. 2). Subsequently, galactose, mannose, arabinose and xylose were being co-fermented. After approximately 30 hours, galactose and mannose were consumed, while after approximately 72 hours, arabinose and xylose were also fully consumed. Both arabinose and xylose contributed significantly to the formation of CO2 and ethanol. However, arabinose and xylose consumption, and thus ethanol and CO2 production slowed down after the exhaustion of galactose and mannose. Strain BIE272 performed generally much better (FIG. 3). Immediately after glucose exhaustion within 10 hours, galactose, mannose, arabinose and xylose were co-fermented rapidly and efficiently. The C5-sugars utilization rates were higher than BIE252. Even after galactose and mannose exhaustion, both pentoses (arabinose and xylose) were co-fermented rapidly, both contributing to CO2-production and ethanol formation. The fermentation of all sugars was completed after approximately 48 hours, in case of strain BIE272. This resulted in a higher cumulative CO₂ and ethanol production and a higher productivity (g ethanol/L/h) of strain BIE272 compared to strain BIE252.

### Example 4

### Performance Test in Real Hydrolysates

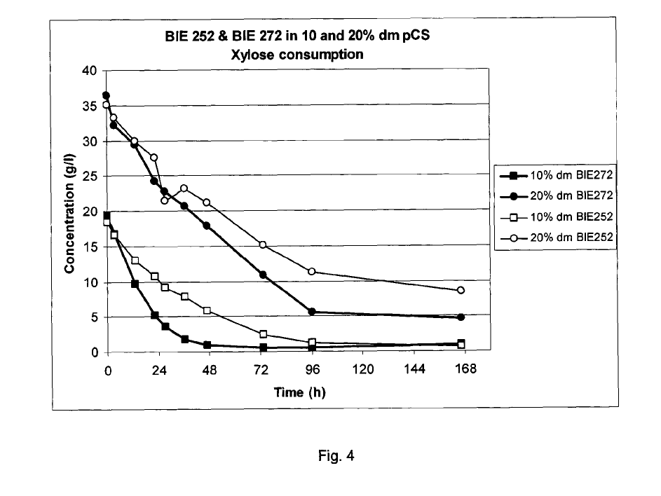

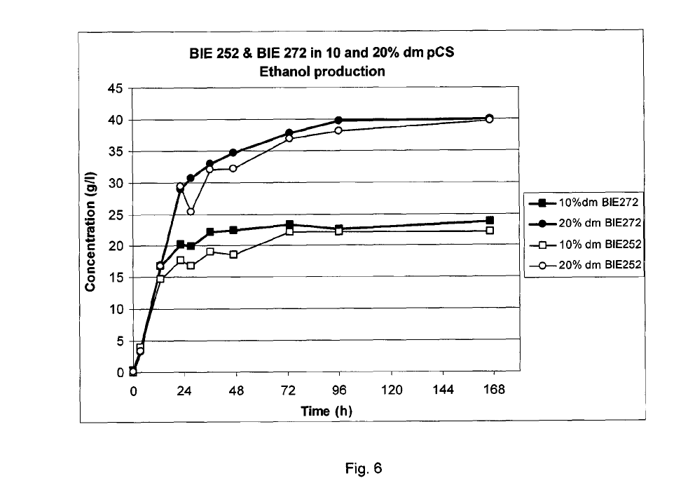

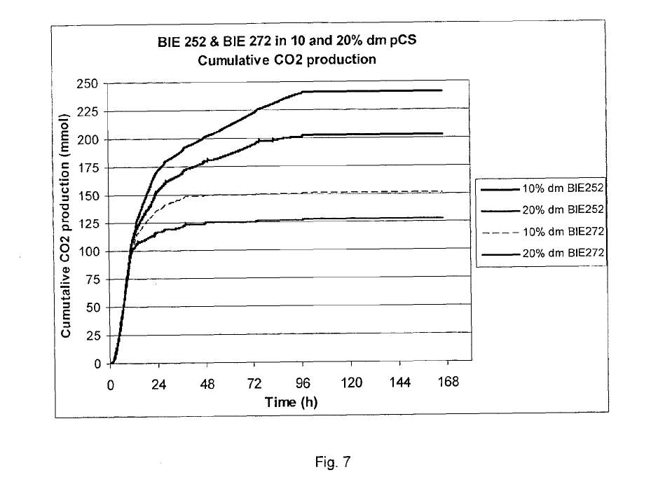

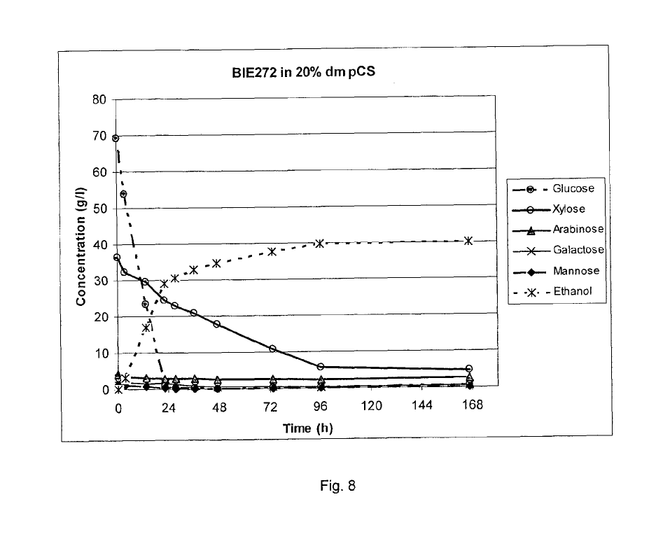

The performance test in real hydrolysates was performed using strains BIE252 and BIE272, which were cultured overnight in shake flasks containing YEP medium supplemented with 2% glucose. The cells were harvested by centrifugation and resuspended at a concentration of 50 grams dry matter per liter. Pretreated corn stover (pCS) at 10 and 20% dry matter were used as feedstocks. The hydrolysis and fermentation were performed as described in the materials and methods section. The results are presented in FIGS. 4 (xylose consumption of BIE252 and BIE272 at 10 and 20% dry matter pCS), 5 (arabinose consumption of BIE252 and BIE272 at 10 and 20% dry matter pCS), 6 (ethanol production of BIE252 and BIE272 at 10 and 20% dry matter pCS) and 7 (CO₂ production of BIE252 and BIE272 at 10 and 20% dry matter pCS). For both strains glucose was readily consumed after the start of the fermentation and the consumption profiles were similar. Glucose was fully consumed after approximately 12 hours at 10% dry matter pCS and after 24 hours at 20% dry matter pCS (data not shown). Strain BIE252 consumed xylose fully in 96 hours at 10% dry matter pCS, while there was still approximately 9 g/l xylose left after 168 hours at 20% dry matter pCS. In case of strain BIE272, xylose was fully consumed in 72 hours at 10% dry matter pCS, while there was less xylose left (5 g/l) compared to strain BIE252 after 168 hours at 20% dry matter pCS (FIG. 4). Arabinose was not fully consumed in case of both strains at both 10% and 20% dry matter. However, in case of strain BIE272, less residual arabinose was measured after 168 h, compared to BIE252 (FIG. 5). Ethanol titer and cumulative CO₂ production were higher in case of strain BIE272 at both 10% and 20% dry matter hydrolysates tested (FIGS. 6 and 7). The overall performance of strain BIE272 at 20% dry matter pCS is presented in FIG. 8. In the table below the yields of the fermentation are calculated, on basis of the sugars liberated at the end of the hydrolysis and the amount of ethanol that was produced at the end of the fermentation.

TABLE 3
Total sugar released (g/l), ethanol produced (g/l) and ethanol
yield (g ethanol/g sugar) of AFM fermentation of BIE252 and BIE272
at 10% and 20% dry matter pretreated corn stover (pCS)

| Lignocel- | Total sugar* | | Produced EtOH | | EtOH yield | |
|---|---|---|---|---|---|---|
| lulosic | (g/l) | | (g/l) | | (g ethanol/g sugar) | |
| feedstock | BIE252 | BIE272 | BIE252 | BIE272 | BIE252 | BIE272 |
| 10% dry | 55.9 | 58.4 | 27.3 | 27.7 | 0.49 | 0.47 |
| matter pCS | | | | | | |
| 20% dry | 108.6 | 113.2 | 45.9 | 48.5 | 0.42 | 0.43 |
| matter pCS | | | | | | |

*(released, monomeric sugar at start fermentation)

### Example 5

### Stability Test of Strain BIE272

5.1 Inoculation Procedure

In order to test the stability of the strain BIE272, 25 μl of a glycerol stock of strain BIE272 was used to inoculated in duplicate flasks in 25 ml of YEP 2% glucose. The optical density of the culture was measured at 600 nm. The cultures were incubated overnight at 30° C. and 280 rpm in a rotary shaker.

After the overnight incubation, the optical densities were determined. Based on the OD⁶⁰⁰ values before and after incubation, the number of generations made during the incubations was calculated. 25 μl of the overnight cultures were used to inoculate a flask containing 25 ml of fresh medium.

The cultures were incubated again under the same conditions as described above. This procedure was repeated until a culture was obtained in which the cells were grown for about 50 generations.

YEP medium supplemented with 2% glucose was chosen, because under these conditions no selection pressure is applied for maintaining the introduced genes and structural variations in strain BIE272, needed for the conversion of arabinose and xylose.

5.2 Isolation of Simile Colonies after Every 9-10 Generations of Growth

In addition to the inoculation procedure described above, the following was applied.

From a glycerol stock of strain BIE272, a loopful of cells was streaked on a YEPD agar plate. The plate was incubated at 30° C. for two days. After two days of incubation, single colonies were visible.

From every overnight culture of section 5.1 (i.e. after 10, 19, 28, 37 or 46 generations), a loopful of cells was streaked on YEPD agar plates in order to generate single colony isolates. The performance of six such colonies was assessed in a growth experiment. To this end the colonies were inoculated in Verduyn medium containing 2% xylose, in 24 well microplates. The plates were incubated for 6 days at 30° C. and 550 rpm in an Infors microplate shaker. The optical density was read at intervals and compared to the initial single colony isolates, directly streaked from the glycerol stock.

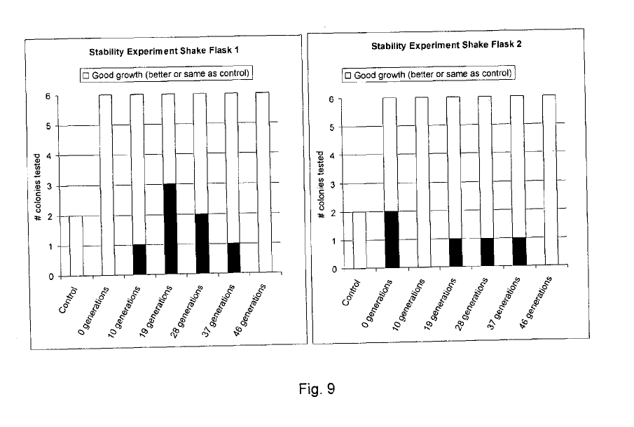

After 48 hours of growth, there was in some cases a difference in growth on xylose between the reference strain BIE272 and the single colony isolates of BIE272 generated after 10, 19, 28, 37 or 46 generations. If the growth of the latter colonies was lagging behind, as indicated by an optical density of 75% or lower as compared to the reference strains, growth was marked as such. The results of the growth at 48 hours of incubation are presented in the FIG. 9.

The results indicate that after about 46 generations, all twelve colonies (six colonies in duplicate) show the same growth phenotype as the colonies of reference strain BIE272 which were isolated directly from the glycerol stock.

After prolonged growth (i.e. beyond 48 hours, up to 6 days, vide supra) all colonies grew confluent, i.e. a maximal optical density was obtained.

The number of generations on the YEPD agar plate was not taken into account. The growth of the single cell, after shake flask incubation and streaked on an agar plate, will grow into a colony on the YEPD agar plate within two days at 30° C. A yeast colony typically has about 3·10⁵-10⁶ cells (Runge, K. W. (2006) Telomeres and Aging in the Yeast Model System. Pages 191-206. In: Handbook of models for human ageing. Edited by P. Michael Conn. ISBN 13: 978-0-12-369391-4). So, starting from one cell to a fully grown colony takes about 18 to 20 divisions, and thus 18-20 additional generations after the culturing in the shake flasks had taken place.

From the results it may be concluded that after more than 50 generations of culturing all colonies exhibited the same growth phenotype as the initial glycerol stock of strain BIE272. Hence, the strain is phenotypically stable.

5.3 Q-PCR Analysis of Xylose Isomerase Genes

In addition to the phenotypic analysis described in section 5.2, a quantitative PCR experiment (Q-PCR) was done in order to assess the copy number of the xylose isomerase genes present in strain BIE272 prior to the cultivation experiment, as well as from the cultures after overnight growth.

After overnight growth, small aliquots of the cultures were used to inoculate fresh medium or to isolate single colonies (see section 5.1 and 5.2). The remainder of the culture was used for the isolation of chromosomal DNA, for Q-PCR purposes.

The Q-PCR analysis was performed using the Bio-Rad iCycler iQ system from Bio-Rad (Bio-Rad Laboratories, Hercules, Calif., USA). The iQ SYBR Green Supermix (Bio-Rad) was used. Experiments were set up as suggested in the manual of the provider.

The stability of the strain BIE272 was assessed by determining the copy number of the xylA gene encoding xylose isomerase. As a reference single copy gene, the ACT1 gene was chosen.

The primers for the detection of the genes xylA and ACT1 are summarized in the table below.

TABLE 4
Primers used for amplification in the Q-PCR experiment

| Gene of interest | Forward primer | Reverse primer |
|---|---|---|
| xylA | SEQ ID NO 1 | SEQ ID NO 2 |
| ACT1 | SEQ ID NO 3 | SEQ ID NO 4 |

The Q-PCR conditions were as follows:

1) 95° C. for 3 min

for 40 cycli, steps 2-4

2) 95° C. for 10 sec

3) 58° C. for 45 sec

4) 72° C. for 45 sec

5) 65° C. for 10 sec

6) Increase of temperature with 0.5° C. per 10 sec to 95° C.

The melting curve is being determined by starting to measure fluorescence at 65° C. for 10 seconds. The temperature is increased every 10 seconds with 0.5° C., until a temperature of 95° C. is reached. From the reads, the copy number of the gene of interest was calculated and/or estimated. The results are presented in the table below.

TABLE 5
Relative copy number of the xylA gene in strain BIE272
and in cultures after 10, 19, 28, 37 and 46 generations
of growth in non-selective YEPD-medium

| Number of | | |
|---|---|---|
| generations | Shake flask 1 | Shake flask 2 |
| 0 | 9 | 9 |
| 10 | 7 | 7 |
| 19 | 13 | 11 |
| 28 | 10 | 11 |
| 37 | 10 | 10 |
| 46 | 8 | 8 |

The values of the xylA-gene copy number, relative to the ACT1 gene, show a small deviation from flask to flask, but are very reproducible when the duplicate flasks are considered.

The results indicated that the copy number of the xylA-gene in BIE272, before and after about 50 generations of cultivation on YEP-medium supplemented with 2% glucose, is essentially the same, taking into account the limitations of the quantitative PCR analysis as previously disclosed by Klein (Klein, D. (2002) TRENDS in Molecular Medicine Vol. 8 No. 6, 257-260). Between 7 and 13 copies of the xylA-gene were detected, in average around 9 copies.

Taken together, the results in this Example show that the strain is phenotypically and genetically stable.

### Example 6

### Resequencing of Selected Strains and Identification of Structural Variations (SV) Involved in Pentose Fermentation

In the Examples described above, it was shown that after adaptive evolution for enhanced growth on the pentoses arabinose and xylose, improved strains could be selected by applying the said selection strategies. Strain BIE272 appeared to be the best strain selected from the single colony isolates with respect to ethanol yield and productivity. Moreover, this strain showed an excellent conversion of mixtures of hexose and pentose sugars, either added to mineral media or present in lignocellulosic hydrolysates, exceeding the performance of pentose fermenting strains known to date.

In addition, it was shown that the selected strains were genetically and phenotypically stable (Example 5). After cultivation on a non-selective medium for about 50 generations, single colony isolates obtained prior and after the cultivations exhibit the same relevant phenotype and genotype.

In order to investigate which genetic variations, such as SNPs (single nucleotide polymorphisms), DIPs (deletion/insertion polymorphisms), deletions, amplifications and rearrangements in the genome of strains BIE252 and BIE272 have contributed to the observed phenotypes (improved ethanol yield and productivity in mixed sugar substrates), we resequenced the genomic DNA of the transformants, using the art known as Solexa® technology, using the Illumina® Genome Analyzer.

To this end, chromosomal DNA was isolated from the strains BIE252 and BIE272 from YEP 2% glucose cultures, previously grown overnight at 280 rpm and 30° C. The DNA was sent to ServiceXS (Leiden, the Netherlands) in case of BIE252 and to BaseClear (Leiden, the Netherlands) in case of BIE272, for resequencing using the Illumina® Genome Analyzer (50 and 75 by reads respectively, paired end sequencing in both cases).

Different sequence yields (i.e. the number of reads) were obtained in the sequencing analysis, mostly related to the state of development in the technology. In all strains, millions of sequence reads were obtained, for example for BIE272, 25 million reads of 75 nucleotides length were obtained, being 1.8 billion nucleotides.

Sequence reads were obtained from the Illumina GAII machine and a quality filtering was applied based on (Phred) quality scores. In addition, low quality and ambiguous nucleotides were trimmed off from the remaining reads.

Using software such as NextGene (SoftGenetics LLC, State College, Pa. 16803, USA) and CLC Genomics workbench v4.5 (CLCbio, Aarhus, Denmark), the sequencing reads were aligned using the S288c as a template in case of strain BIE252. In case of BIE272, alignment was done using CLC Genomics workbench v4.5 to the template of S288c and in a second analysis, an assembly of previously obtained sequencing data of strain BIE104 was used as a reference template.

The average read depth per base that was obtained after mapping the reads to the template:
BIE252: read depth of 121
BIE272: read depth of 135

Mutations (single nucleotide polymorphisms and insertion/deletions up to 30 bp) were detected and summarised in a mutation report. The mutations called in the different strains were compared to each other to identify the unique variations between the strains.

Every entry of the mutation report was checked manually, in order to rule out the possibility of misalignment of the sequence reads, or mutation calls due to sequencing errors or calls for which sequence coverage was very low. False positive mutations were removed from the mutation report.

Table 7 presents an overview of the SNPs that were observed.

TABLE 7
Single nucleotide polymorphisms (SNPs) in strains BIE252 and BIE272, relative to strain BIE104.
For reference, the structural variations in strain BIE201 have been listed as well.

| | | | | Amino acid | | | | |
|---|---|---|---|---|---|---|---|---|
| ORF | Gene | Chromosome | SNP | change | BIE104 | BIE201 | BIE252 | BIE272 |
| YDR160w | SSY1 | IV | G > T | Glu455STOP | G | T | T | T |
| | | | | (>stop codon) | | | | |
| YJR154w | | X | A > G | Asp171Gly | A | G | G | G |
| YMR168c | CEP3 | XIII | A > G | Ser396Gly | A | G | G | G |
| YML051w | GAL80 | XIII | A > C | Thr146Pro | A | C | C | C |
| YGL167C | PMR1 | VII | A > G | Tyr38Cys | A | A | A | G |

In addition, using the coverage plots indicating the read depth at every single nucleotide position of the genome, searches were done for areas in the genome that are over- or underrepresented. Such structural variations comprise deletions, duplications, copy number variations, insertions, inversions and translocations.

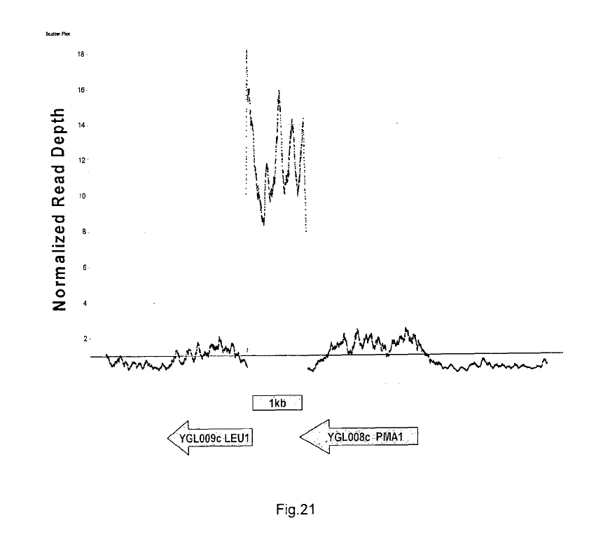

FIG. 21 sets out an example of an increased coverage of the PMA1 terminator region. This terminator has been used in several constructs for the overexpression of the genes araA, araB and araD (in plasmid pPWT018 (see PCT/EP2011/056242) as well as the xylA-gene (see EP10160647.3)). As a consequence, multiple copies of the PMA1 terminator are present in the genome of strains BIE252 and BIE272, resulting in an increased read depth as compared to the surrounding genomic regions of the PMA1 terminator.

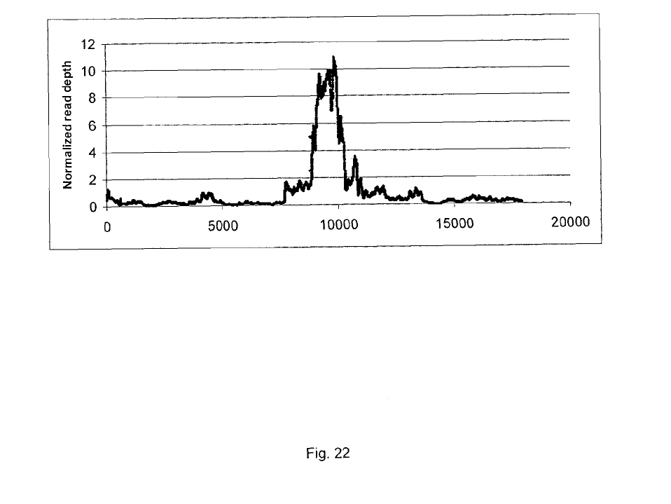

FIG. 22 sets out another example of a coverage analysis, in this case of the xylA gene encoding xylose isomerase. The normalized read depth of the region consisting of the xylA gene corresponds with a value of 9 to 10, which is in line with the copy number as determined by Q-PCR (see Example 5), while the read depth of the surrounding genomic regions is around 1.

Using this method, regions that were over- and underrepresented in strains BIE252 and BIE272 were identified. It was found that the amplification previously observed in strain BIE201 (see PCT/EP2011/056242), based on the information now available, is located on the left arm of chromosome VII, and that left arm is no longer amplified in strains BIE252 and BIE272. The amplification observed on the right arm of chromosome VII in BIE201, comprising the genes araA, araB and araD, are conserved in strains BIE252 and BIE272. The copy number of the arabinose genes in BIE272 was determined as three copies, based on the normalized read depth.

### Example 7

### Analysis of the Chromosome Structure of Transformants

From resequencing data (see Example 6) it could be inferred that after adaptive evolution, genome variations in the genome have taken place. These genome variations include single nuclear polymorphisms (SNPs), deletion-insertion polymorphisms (DIPs) and larger variations in the structure of the chromosomes, due to events like amplification and translocation. In order to substantiate the latter, CHEF gel electrophoresis was employed.

Contour-clamped homogeneous electric field (CHEF) gel electrophoresis has been used to study the karyotypes of a range of Saccharomyces cerevisiae yeast strains, from the untransformed strain BIE104 up to strain BIE272, a strain that is able to ferment pentoses and hexoses rapidly in sugar mixtures.

7.1 CHEF Electrophoresis

In order to determine whether the number and the size of the chromosomes were changed, or the composition with respect to certain key genes, CHEF electrophoresis (Clamped Homogeneous Electric Fields electrophoresis; CHEF-DR® Ill Variable Angle System; Bio-Rad, Hercules, Calif. 94547, USA) was applied. Agarose plugs of yeast strains (see below) were prepared using the CHEF Yeast Genomic DNA Plug Kit (BioRad) according to the instructions of the supplier. 1% Agarose gels (Pulse Field Agarose, Bio-Rad) were prepared in 0.5×TBE (Tris-Borate-EDTA) according to the suppliers instructions. Gels were run according to the following settings:

Block 1 initial time 60 sec
final time 80 sec
ratio 1
run time 15 hours

Block 2 initial time 90 sec
final time 120 sec
ratio 1
run time 9 hours

As a marker for size determination of the chromosomes, agarose plugs of strain YNN295 (Bio-Rad) were included in the experiment.

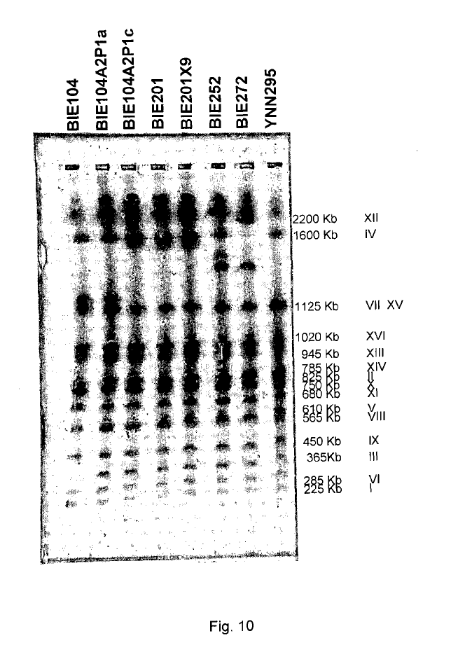

After electrophoresis, gels were stained using ethidiumbromide at a final concentration of 70 μg per litre, for 30 minutes. In FIG. 10, an example of a stained gel is shown.

In strains BIE104A2P1c, BIE201 and BIE201X9, the size of chromosome VII was increased. Its size was increased to a size close to that of chromosome IV, of about 1500-1550 kb.

In strain BIE252 however, the large size of chromosome VII was decreased as compared to BIE201, but still larger than the original size of chromosome VII (as it is in strain BIE104, the untransformed yeast strain). Two chromosomes appeared, with a size of approximately 1375 and 1450 kb respectively. This result corroborates the observation from the resequencing data (Example 6), that the left arm of chromosome VII, which was amplified in strain BIE201, is no longer amplified in strains BIE252 and BIE272. Since the amplifications on the right arm of chromosome VII are still present in BIE252 and BIE272, as in BIE201, the size of chromosome VII is still larger than the original size of chromosome VII (as it appears in BIE104).

In strain BIE272, the situation seems even more complex. The 1600 kb band, representing chromosome IV, is no longer visible. Since on chromosome IV, like on any other chromosome, several indispensible genes are present, the chromosome size presumably has been changed by either fragmentation (i.e. split in two smaller parts) or increase in size (e.g. by amplification).

In addition, the chromosome with a size of approximately 1450 kb has disappeared. The chromosome with a size of approximately 1375 kb is also present in BIE272.

One way of identifying how the chromosomes have been rearranged, either by amplification of parts, translocation and/or fragmentation, is to transfer the DNA of the gel by blotting followed by hybridisation with specific probes, which are representative for certain chromosomes.

7.2 Hybridisation with Specific Probes

After staining, gels were blotted onto Amersham Hybond N+ membranes (GE Healthcare Life Sciences, Diegem, Belgium).

In order to be able to identify the nature of the changes in the size of the chromosomes, probes were made for hybridization with the blotted membranes. Probes (see table below) were prepared using the PCR DIG Probe Synthesis Kit (Roche, Almere, the Netherlands) according to the instructions of the supplier.

The following probes were prepared.

TABLE 8
Primers for amplification of the indicated probes

| | Systematic | Forward | Reverse | Size PCR | Chromo- |
|---|---|---|---|---|---|
| Probe | name gene | primer | primer | product (bp) | some |
| xylA | | SEQ ID | SEQ ID | 419 | V + other |
| | | NO 1 | NO 2 | | chromo- |
| | | | | | some(s) |
| ACT1 | YFL039c | SEQ ID | SEQ ID | 392 | VI |
| | | NO 3 | NO 4 | | |
| PNC1 | YGL037c | SEQ ID | SEQ ID | 384 | VII |
| | | NO 5 | NO 6 | | |

Membranes were prehybridized in DIG Easy Hyb Buffer (Roche) according to the instructions of the supplier. The probes were denatured at 99° C. for 5 minutes, chilled on ice for 5 minutes, and added to the prehybridized membranes. Hybridization was done overnight at 42° C.

Washing of the membranes and blocking of the membranes prior to detection of the hybridized probes were done using the DIG Wash and Block Buffer Set (Roche) according to the instructions of the supplier. The detection was done by incubation with anti-dioxygenin-AP Fab fragments (Roche) followed by the addition of detection reagents using the CDP-Star ready-to-use kit (Roche). Detection of the chemiluminiscent signals were performed using the Bio-Rad Chemidoc XRS+System, using the appropriate settings provided by the Chemidoc apparatus.

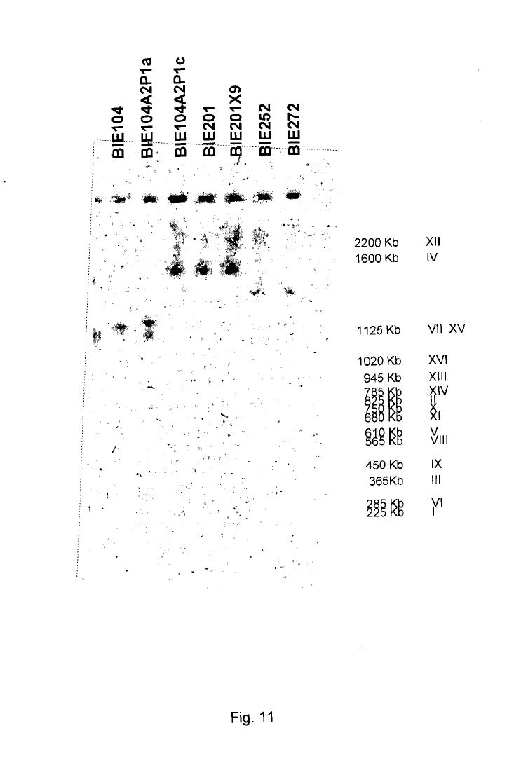

The results are shown in FIGS. 11 (PNC1), 12a (ACT1) and 12b (xylA).

PNC1 is located on the left arm of chromosome VII, and thus considered to be a specific probe for this chromosome. Hybridization resulted in a band of the expected size in case of strain BIE104, the untransformed strain. In strain BIE104A2P1 (designated in FIG. 11 as BIE104A2P1a), the same band is observed. In addition, a second more faint and smaller band is observed. The corresponding band is absent in the ethidiumbromide stained gel (FIG. 10). Hence, this signal is probably an electrophoresis (trapping) and or a hybridization artefact.

In strains BIE104A2P1c, BIE201 and BIE201X9, an increase in the size of chromosome VII is observed, as was apparent from the ethidiumbromide stained gel (FIG. 10). Its size was increased to a size close to that of chromosome IV, of about 1500-1550 kb.

In strains BIE252 and BIE272, a band of smaller size hybridized. The size is approximately 1375 kb. In BIE252, a second, larger but less intense band is observed, which is absent in BIE272. This band may be the result of an electrophoresis (trapping) and/or a hybridization artefact. Alternatively, it is a larger form of the same chromosome. Since the agarose plugs were prepared from a purified single colony isolate, this is not very likely.

Based on intensity comparisons, these results corroborate the observations described above that the left arm of chromome VII is no longer amplified in strains BIE252 and BIE272. From the intensity of the bands, it can be deduced that the copy number of the PNC1 gene is increased in strains BIE104A2P1c, BIE201 and BIE201X9 relative to BIE104 and BIE104A2P1(a), but decreased again in strains BIE252 and BIE272.

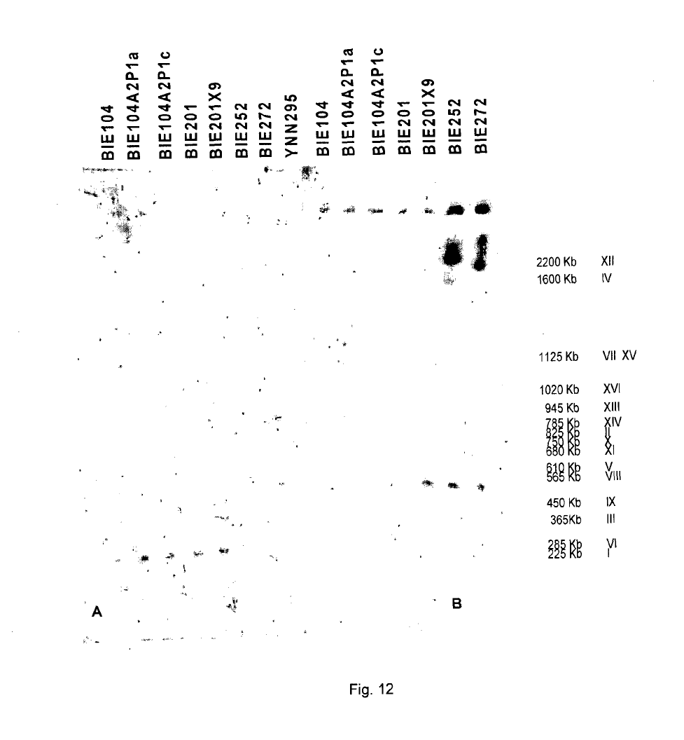

The ACT1-gene is located on chromosome VI and not expected to be amplified. Hence, this probe serves as a control. Indeed, a single band was observed after hybridisation (see FIG. 12, panel a) in all strains tested.

The xylA-gene was integrated as a single copy gene on chromosome V in strain BIE201X9. In strain BIE252, extra copies were introduced in the Ty1 loci of strain BIE201X9, followed by adaptive evolution, finally yielding strain BIE272.

As expected, in strain BIE201X9, one single chromosome hybridizes with the xylA-probe. The band observed on the autoradiogram has a size of approximately 600 kb, which is the right size. Please note that in this genetic background the resolution between chromosomes V and VIII is less pronounced as is the case in the marker strain, YNN295 (see FIG. 10).

The same band is observed in the strains BIE252 and BIE272.

In strain BIE252, at least one extra band is observed which has a high molecular weight. The band is around 2 Mb, which suggests that the integration of the extra copies of the xylA-gene have taken place on chromosome XII, which is the largest chromosome. The intensity of the band is high in comparison to the intensity of the band corresponding to the integrated xylA-gene on chromosome V. From the ratio of the intensities of both bands, the copy number may be inferred. It may be concluded that multiple copies of the xylA-gene have been integrated in chromosome XII. The exact determination of the copy number requires more elaborate work, such as the application different concentrations of DNA, and the application of densitometry (to quantify the DNA by measuring the density of silver grains on the photograph) of autoradiograms with several exposure times, in order to assure that the readings obtained are within the linear range of the film. In addition, although an increase in the signal intensity may suggest an increase of the copy number of a certain gene, other factors may also influence the signal strength, like the amount of DNA applied on the gel, blotting efficiency, detection saturation, and the like.

Two extra, fainter bands were observed in strain BIE252, one being slightly higher and one being slightly smaller than the most intense band. Presumably, these bands are electrophoresis artefacts caused by trapping of DNA.

In strain BIE272, the size of the strongest hybridizing band has decreased in size as compared to strain BIE252, suggesting a structural variation of chromosome XII (FIG. 12b). This is also observed in the ethidiumbromide stained gel (FIG. 10). Also in case of strain BIE272, “shadowbands” occur which are most likely due to trapping of chromosomes during electrophoresis. The intensity of the band corresponding to chromosome XII is several times higher than the intensity of the band corresponding to chromosome V, suggesting that multiple copies of the xylA-gene are still present in strain BIE272, as was observed for strain BIE252. From the Q-PCR experiments it was concluded that around 9 copies of the xylA-gene are present in strain BIE272 (Example 5, section 5.3).

In the stained gel (FIG. 10), the band corresponding to chromosome IV is no longer visible in strain BIE272, suggesting a recombinational event in which chromosome IV is involved.

In conclusion, the results of Example 7 clearly indicate that structural variations leading to shifts in chromosome sizes have taken place. More elaborate studies will be needed in order to be able to conclude which (parts of) chromosomes were involved in these processes.

### Example 8

### Performance Test of the Strains BIE104, BIE201, BIE252 and BIE272

The strains BIE104, BIE201, BIE252 and BIE272 have different characteristics with respect to their genetic constitution and their performance in sugar hydrolysates. The table below illustrates how the strains relate to each other.

TABLE 9
Relevant strains in the strain lineage of BIE272

| | | | Adaptive |
|---|---|---|---|
| Strain | Parent strain | Genes introduced | evolution |
| BIE104 | Wild-type strain | | |
| BIE201 | BIE104 | TAL1, TKL1, RPE1, RKI1, | In shake flasks |
| | | araA, araB, araD | |
| BIE252 | BIE201 | xylA, XKS1 | In shake flasks |
| BIE272 | BIE252 | None | In fermentors |

In order to illustrate the improvements with respect to the conversion of sugar mixtures that were achieved during the development of strain BIE272, a performance test was executed in the AFM (Halotec, Vennendaal, the Netherlands). To this end, single colony isolates of the strains BIE104, BIE201, BIE252 and BIE272 were cultivated in 100 ml Verduyn medium with 2% glucose as the carbon source, for 24 hours at 30° C. and 280 rpm.

The cells were harvested by centrifugation and cultivations for CO2 production were performed at 33° C. and pH 4.2 in 200 ml Verduyn medium supplemented with 50 g/l glucose, 50 g/l xylose, 35 g/l arabinose, 10 g/l galactose and 5 g/l mannose in the AFM (temperature 33° C., stirrer speed 250 rpm, fermentation time minimally 72 hours). The CO₂ production was constantly monitored at intervals, and samples were taken for analysis (optical density at 600 nm using a spectrophotometer; ethanol, glycerol and residual sugars by NMR).

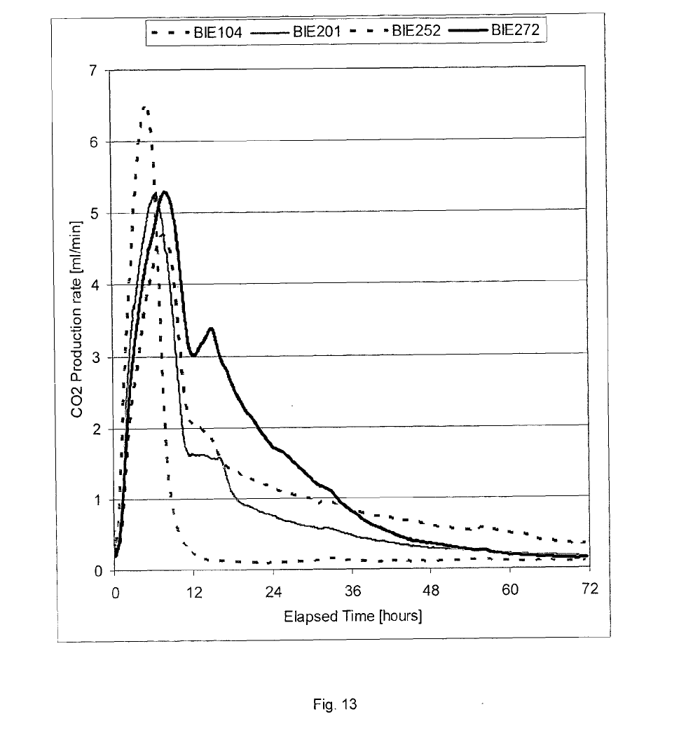

The CO₂ evolution profiles are set out in FIGS. 13, 14, 15 and 16. The total amount of CO2 that was produced during the experiment, which lasted 71 hours and 25 minutes, is set out in table 10.

TABLE 10
Total amount of CO2 produced by strains BIE104, BIE201,
BIE252 and BIE272 in Verduyn medium containing 50 g/l
glucose, 50 g/l xylose, 35 g/l arabinose, 10 g/l galactose
and 5 g/l mannose, in about 72 hours.

| Strain | Amount of CO₂ produced (ml) |
|---|---|
| BIE104 | 2478 |
| BIE201 | 4208 |
| BIE252 | 5108 |
| BIE272 | 6066 |

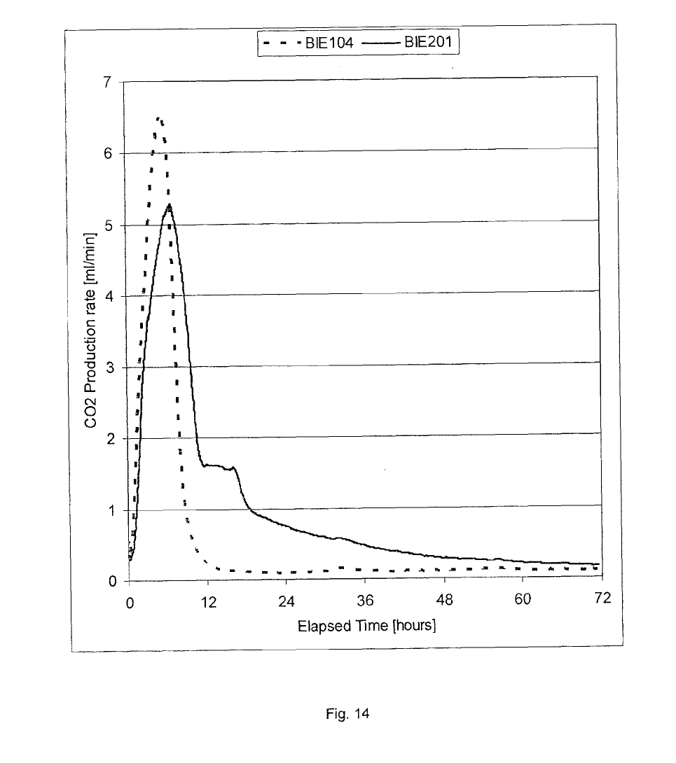

In FIG. 13, all four strains are shown in one graph. In the FIGS. 14, 15 and 16, a pairwise comparison of two strains at the time is made.

In FIG. 14, strain BIE104 is compared to BIE201. From this sugar mixture, strain BIE104 can only ferment glucose and mannose, while strain BIE201 ferments glucose, mannose, galactose and arabinose. This resulted in a different CO₂ production rate profile (FIG. 14) and an increase of 70% in the total amount of CO₂ produced.

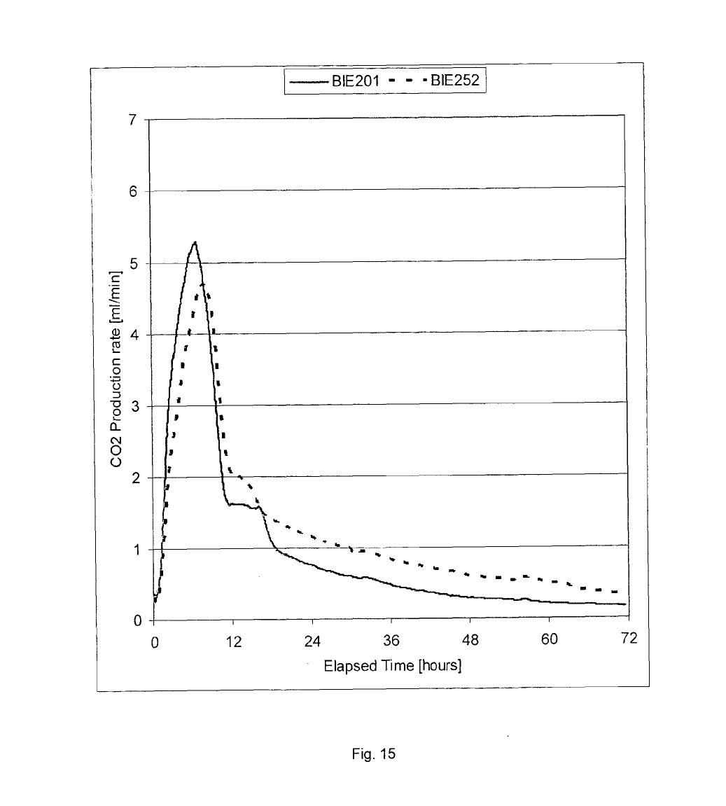

In FIG. 15, strains BIE201 and BIE252 were compared. The ability to convert xylose, which is new in strain BIE252 relative to BIE201, next to arabinose and the hexoses, yielded a higher CO₂ production rate (FIG. 15) and a higher total CO₂ production (+106% relative to BIE104 and +21% relative to BIE201).

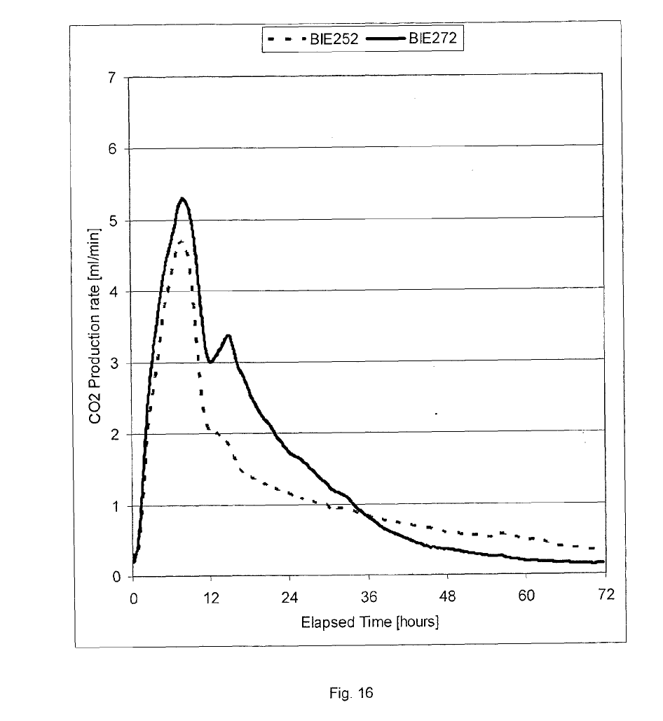

In FIG. 16, it is shown that strain BIE272 showed a higher conversion rate of sugars into ethanol and carbondioxide. At the end of the experiment (71 hours and 25 minutes), strain BIE272 had produced 145% more CO₂ relative to BIE104, and 19% more relative to strain BIE252.

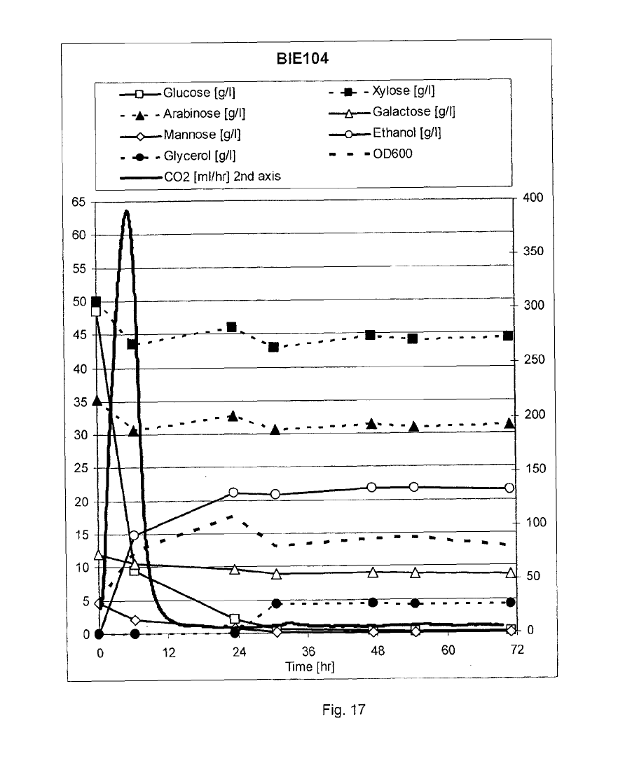

In FIGS. 17, 18, 19 and 20, the sugar consumption and ethanol formation are set out for strains BIE104, BIE201, BIE252 and BIE272 respectively.

Strain BIE104 (FIG. 17) only consumes the glucose and mannose. The strain is not capable of converting the pentoses xylose and arabinose, since this is a non-transformed strain. Also galactose is not converted, for under fermentative conditions, the energy charge was probably too low to allow synthesis of the Leloir proteins for galactose utilization, as described by van den Brink et al (van den Brink et al (2009) Energetic limits to metabolic flexibility: responses of Saccharomyces cerevisiae to glucose-galactose transitions. Microbiology 155(Pt 4):1340-50). The yield on dosed sugars amounts 0.14 grams of ethanol per gram sugar, in 72 hours.

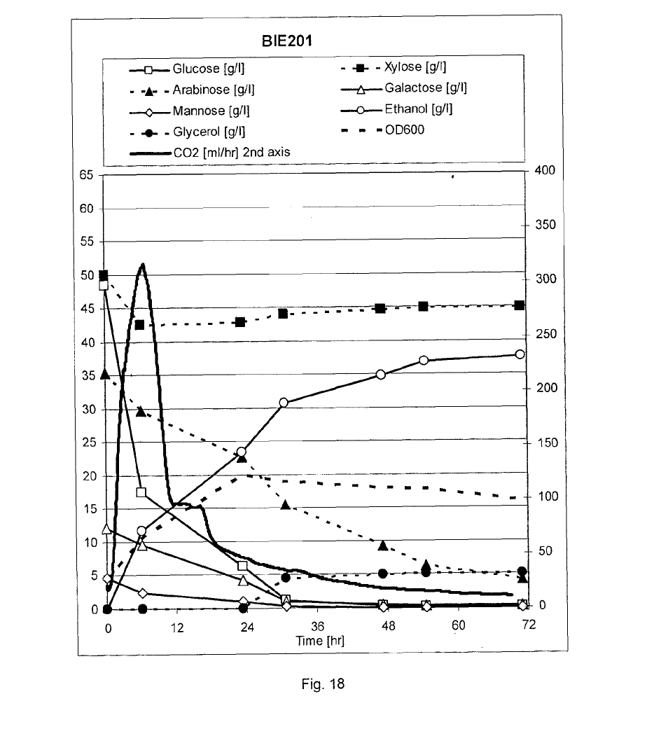

Strain BIE201 (FIG. 18) is capable of converting glucose, mannose, arabinose and galactose. Xylose is not fermented, since the pathway for xylose fermentation was not introduced in this strain. In 72 hours, arabinose is almost completely fermented in this experiment, while the hexoses, including galactose, were completely converted before 36 hours after the start of the experiment. The yield on dosed sugars amounts 0.25 grams of ethanol per gram sugar, in 72 hours.

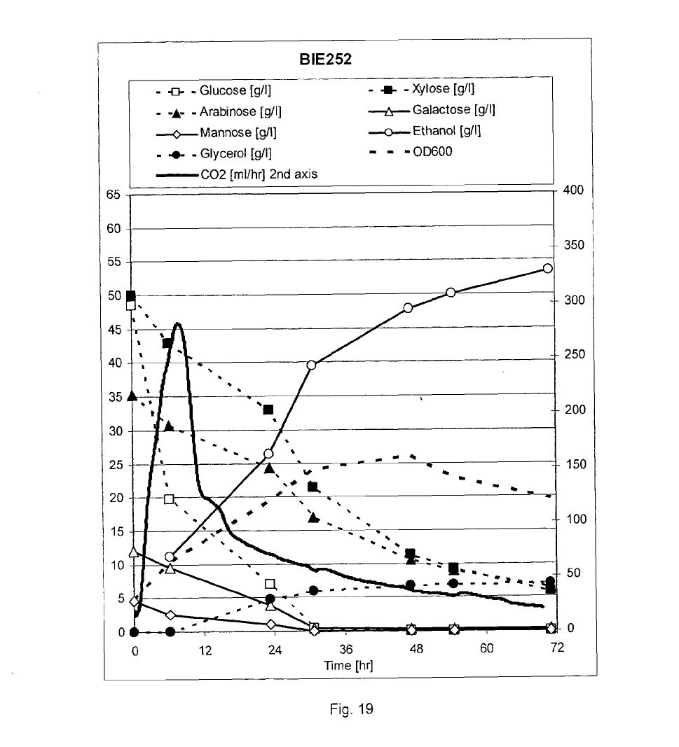

Strain BIE252 (FIG. 19) is capable of fermenting glucose, xylose, mannose, arabinose and galactose. In 72 hours, xylose and arabinose are almost completely fermented in this experiment, while the hexoses glucose, mannose and galactose were already exhausted before 36 hours after the start of the experiment. The yield on dosed sugars amounts 0.36 grams of ethanol per gram sugar, in 72 hours.

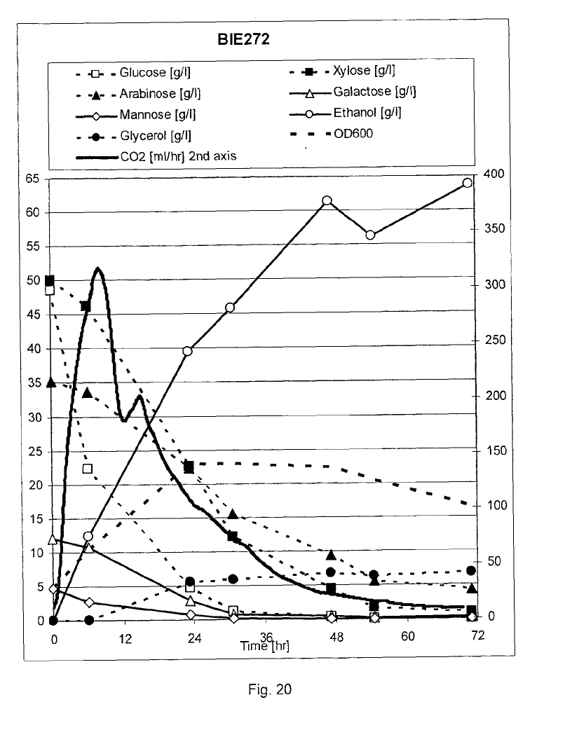

Strain BIE272 (FIG. 20) is capable of fermenting glucose, xylose, mannose, arabinose and galactose. In 72 hours, all sugars were fermented rapidly and completely, except for arabinose, which was fermented almost completely. The yield on dosed sugars amounts 0.42 grams of ethanol per gram sugar, in 72 hours.

The fermentation characteristics of strains BIE104, BIE201, BIE252 and BIE272 are summarized in the table below.

TABLE 11
Fermentation characteristics of strains BIE104, BIE201, BIE252
and BIE272 in Verduyn medium containing 5% glucose, 5% xylose,
3.5% arabinose, 1% galactose and 0.5% mannose. The yield is
expressed as grams of ethanol per gram dosed sugar, calculated
over the whole fermentation (72 hours). The productivity is
expressed as grams of ethanol per liter per hour, calculated
over the time period indicated in the table.

| | | Produc- | Produc- | Produc- | Produc- |
|---|---|---|---|---|---|
| | | tivity | tivity | tivity | tivity |
| | Yield | 0-24 h | 24-48 h | 0-48 h | 0-72 h |
| | (g EtOH/ | (g EtOH/ | (g EtOH/ | (g EtOH/ | (g EtOH/ |
| Strain | g sugar) | l, h) | l, h) | l, h) | l, h) |
| BIE104 | 0.14 | 0.91 | 0.03 | 0.46 | 0.30 |
| BIE201 | 0.25 | 1.01 | 0.47 | 0.74 | 0.53 |
| BIE252 | 0.36 | 1.13 | 0.90 | 1.01 | 0.75 |
| BIE272 | 0.42 | 1.69 | 0.91 | 1.30 | 0.90 |

As is clear from table 11, not only the yield was increased in case of strain BIE272 in the overall process, but also the productivity at several time intervals. In the first 24 hours of the fermentation, the productivity was increased by 50% in case of strain BIE272, as compared to strain BIE252.

In addition to the increased yield and productivity of strain BIE272 relative to the other strains tested, the consumption rate of xylose and arabinose, in the presence of glucose, was increased as well.

At time point 6.2 hours, the culture of strain BIE252 contained 19.8 g glucose per liter (110 mM). At time point 23.3 hours, the glucose concentration was 7.1 g/l (39 mM). Both concentrations are repressing concentrations (glucose or catabolite repression), at which the use of other carbon sources than glucose is actively prevented. Surprisingly, during this time period, the arabinose concentration decreased from 30.8 g/l to 24.3 g/l and the xylose concentration decreased from 42.8 g/l to 32.9 g/l. So, co-consumption of glucose, xylose and arabinose took place under these conditions.

In case of strain BIE272, the following decreases were observed from time point 6.2 hours until 23.3 hours: glucose from 22.4 g/l to 4.8 g/l (from 124 mM to 27 mM), arabinose from 33.7 g/l to 22.4 g/l and xylose from 46.2 g/l to 22.6 g/l. Also in this strain, co-consumption of xylose, arabinose and glucose took place.

The consumption rates of arabinose and xylose, in the presence of glucose, were calculated as grams of pentose consumed per hour per gram dry yeast biomass. The values are presented in table 12.

TABLE 12
Consumption rates of arabinose and xylose in the
presence of glucose of strains BIE252 and BIE272

| | Arabinose consumption rate | Xylose consumption rate |
|---|---|---|
| Strain | (g/g, h) | (g/g, h) |
| BIE252 | 0.13 | 0.19 |
| BIE272 | 0.20 | 0.41 |

The ability to consume the pentose arabinose and xylose in the presence of repressing glucose concentrations was further improved in strain BIE272.

1. A yeast cell belonging to the genus Saccharomyces having introduced into the genome thereof, at least one xylA gene and at least one of each of araA, araB and araD genes and said yeast cell being capable of consuming a mixed sugar mixture comprising glucose, xylose and arabinose, wherein the cell co-consumes glucose and arabinose, and comprises genetic variations obtained during adaptive evolution and comprises a specific xylose consumption rate in the presence of glucose that is at least 0.25 g xylose/h, g DM.

2. The yeast cell according to claim 1, wherein said yeast cell is Saccharomyces cerevisiae.

3. The yeast cell according to claim 1, wherein the specific xylose consumption rate in the presence of glucose is at least 0.35 g xylose/h, g DM.

4. The yeast cell according to claim 1, wherein the specific xylose consumption rate in the presence of glucose is at least from 0.25 to 0.60 g xylose/h, g DM.

5. The yeast cell according to claim 1, wherein copy numbers of the araA, araB and araD genes are three or four each.

6. The yeast cell according to claim 1, wherein the copy number of xylA is about 9 or 10.

7. The yeast cell according to claim 1, having at least one single nucleotide polymorphism selected from the group consisting of mutations G1363T in the SSY1 gene, A512T in YJR154w gene, A1186G in CEP3 gene, A436C in GAL80 gene and A113G in PMR1 gene.

8. The yeast cell according to claim 6, which comprises a single polymorphism A436C in GAL80 gene.

9. The yeast cell according to claim 6, which comprises a single nucleotide polymorphism A1186G in CEP3 gene.

10. The yeast cell according to claim 6, which comprises a single nucleotide polymorphism A113G in PMR1 gene.

11. The yeast cell according to claim 1, wherein said yeast cell comprises a yield of at least 0.40 g ethanol/g sugars or about 0.42 g ethanol/g sugars.

12. The yeast cell according to claim 1, wherein said yeast cell comprises a productivity of at least 1.20 g EtOH/1, h or about 1.69 g EtOH/1, h, measured in an interval of from 0 to 24 h after start of fermentation.

13. A polypeptide comprising the sequence SEQ ID NO: 8 and variant polypeptides thereof, wherein at least one other position may comprise mutation of an aminoacid with an aminoacid that is an existing conserved aminoacid in the SPCA family.

14. A process for producing at least one fermentation product from a sugar composition comprising glucose, galactose, arabinose and xylose, wherein said sugar composition is fermented with a yeast cell according to claim 1.

15. The process according to claim 14, wherein the sugar composition is produced from lignocellulosic material by:
a) pretreatment of at least one lignocellulosic material to produce pretreated lignocellulosic material;
b) enzymatic treatment of the pretreated lignocellulosic material to produce the sugar composition.

16. The process according to claim 14, wherein fermentation is conducted anaerobically.

17. The process according to claim 14, wherein the fermentation product is selected from the group consisting of ethanol, n-butanol, isobutanol, lactic acid, 3-hydroxy-propionic acid, acrylic acid, acetic acid, succinic acid, fumaric acid, malic acid, itaconic acid, maleic acid, citric acid, adipic acid, an amino acid, such as lysine, methionine, tryptophan, threonine, and aspartic acid, 1,3-propane-diol, ethylene, glycerol, a β-lactam antibiotic and a cephalosporin, vitamins, pharmaceuticals, animal feed supplements, specialty chemicals, chemical feedstocks, plastics, solvents, fuels, including biofuels and biogas or organic polymers, and an industrial enzyme, optionally comprising a protease, a cellulase, an amylase, a glucanase, a lactase, a lipase, a lyase, an oxidoreductases, a transferase or a xylanase.


SEQUENCE LISTING (8 sequences)

SEQ ID NO: 1 - DNA, 22 bp, Artificial Sequence
  synthetic DNA, forward primer xylA
caccgttagccttggcgtaagc

SEQ ID NO: 2 - DNA, 21 bp, Artificial Sequence
  synthetic DNA, reverse primer xylA
cactttcgaacacgaattggc

SEQ ID NO: 3 - DNA, 21 bp, Artificial Sequence
  synthetic DNA, forward primer ACT1
gttacgtcgccttggacttcg

SEQ ID NO: 4 - DNA, 21 bp, Artificial Sequence
  synthetic DNA, reverse primer ACT1
cggcaatacctgggaacatgg

SEQ ID NO: 5 - DNA, 22 bp, Artificial Sequence
  synthetic DNA, forward primer PNC1
gatagagactggcacaggattg

SEQ ID NO: 6 - DNA, 21 bp, Artificial Sequence
  synthetic DNA, reverse primer PNC1
acaatactccaaagctacacc

SEQ ID NO: 7 - protein, 950 aa, Saccharomyces cerevisiae
MSDNPFNASLLDEDSNREREILDATAEALSKPSPSLEYCTLSVDEALEKLDTDKNGGLRSSNEANNRRSLYGPNEITVEDDESLFKKFLSNFIEDRMILLLIGSAVVSLFMGNIDDAVSITLAIFIVVTVGFVQEYRSEKSLEALNKLVPAECHLMRCGQESHVLASTLVPGDLVHFRIGDRIPADIRIIEAIDLSIDESNLTGENEPVHKTSQTIEKSSFNDQPNSIVPISERSCIAYMGTLVKEGHGKGIVVGTGTNTSFGAVFEMMNNIEKPKTPLQLTMDKLGKDLSLVSFIVIGMICLVGIIQGRSWLEMFQISVSLAVAAIPEGLPIIVTVTLALGVLRMAKRKAIVRRLPSVETLGSVNVICSDKTGTLTSNHMTVSKLWCLDSMSNKLNVLSLDKNKKTKNSNGNLKNYLTEDVRETLTIGNLCNNASFSQEHAIFLGNPTDVALLEQLANFEMPDIRNTVQKVQELPFNSKRKLMATKILNPVDNKCTVYVKGAFERILEYSTSYLKSKGKKTEKLTEAQKATINECANSMASEGLRVFGFAKLTLSDSSTPLTEDLIKDLTFTGLIGMNDPPRPNVKFAIEQLLQGGVHIIMITGDSENTAVNIAKQIGIPVIDPKLSVLSGDKLDEMSDDQLANVIDHVNIFARATPEHKLNIVRALRKRGDVVAMTGDGVNDAPALKLSDIGVSMGRIGTDVAKEASDMVLTDDDFSTILTAIEEGKGIFNNIQNFLTFQLSTSVAALSLVALSTAFKLPNPLNAMQILWINILMDGPPAQSLGVEPVDHEVMKKPPRKRTDKILTHDVMKRLLTTAACIIVGTVYIFVKEMAEDGKVTARDTTMTFTCFVFFDMFNALACRHNTKSIFEIGFFTNKMFNYAVGLSLLGQMCAIYIPFFQSIFKTEKLGISDILLLLLISSSVFIVDELRKLWTRKKNEEDSTYFSNV

SEQ ID NO: 8 - protein, 950 aa, Saccharomyces cerevisiae
MSDNPFNASLLDEDSNREREILDATAEALSKPSPSLECCTLSVDEALEKLDTDKNGGLRSSNEANNRRSLYGPNEITVEDDESLFKKFLSNFIEDRMILLLIGSAVVSLFMGNIDDAVSITLAIFIVVTVGFVQEYRSEKSLEALNKLVPAECHLMRCGQESHVLASTLVPGDLVHFRIGDRIPADIRIIEAIDLSIDESNLTGENEPVHKTSQTIEKSSFNDQPNSIVPISERSCIAYMGTLVKEGHGKGIVVGTGTNTSFGAVFEMMNNIEKPKTPLQLTMDKLGKDLSLVSFIVIGMICLVGIIQGRSWLEMFQISVSLAVAAIPEGLPIIVTVTLALGVLRMAKRKAIVRRLPSVETLGSVNVICSDKTGTLTSNHMTVSKLWCLDSMSNKLNVLSLDKNKKTKNSNGNLKNYLTEDVRETLTIGNLCNNASFSQEHAIFLGNPTDVALLEQLANFEMPDIRNTVQKVQELPFNSKRKLMATKILNPVDNKCTVYVKGAFERILEYSTSYLKSKGKKTEKLTEAQKATINECANSMASEGLRVFGFAKLTLSDSSTPLTEDLIKDLTFTGLIGMNDPPRPNVKFAIEQLLQGGVHIIMITGDSENTAVNIAKQIGIPVIDPKLSVLSGDKLDEMSDDQLANVIDHVNIFARATPEHKLNIVRALRKRGDVVAMTGDGVNDAPALKLSDIGVSMGRIGTDVAKEASDMVLTDDDFSTILTAIEEGKGIFNNIQNFLTFQLSTSVAALSLVALSTAFKLPNPLNAMQILWINILMDGPPAQSLGVEPVDHEVMKKPPRKRTDKILTHDVMKRLLTTAACIIVGTVYIFVKEMAEDGKVTARDTTMTFTCFVFFDMFNALACRHNTKSIFEIGFFTNKMFNYAVGLSLLGQMCAIYIPFFQSIFKTEKLGISDILLLLLISSSVFIVDELRKLWTRKKNEEDSTYFSNV